SEQUENCE LISTING (34 sequences)

SEQ ID NO: 1 - protein, 224 aa, Pyrococcus abyssi
MKVAGADEAGRGPVIGPLVIVAAVVEEDKIRSLTKLGVKDSKQLTPAQREKLFDEIVKVLDDYSVVIVSPQDIDGRKGSMNELEVENFVKALNSLKVKPEVIYIDSADVKAERFAENIRSRLAYEAKVVAEHKADAKYEIVSAASILAKVIRDREIEKLKAEYGDFGSGYPSDPRTKKWLEEWYSKHGNFPPIVRRTWDTAKKIEEKFKRAQLTLDNFLKRFRN

SEQ ID NO: 2 - protein, 245 aa, Artificial Sequence
  Recombinant Pyrococcus abyssi RNase H2
AMDIGINSDPMKVAGADEAGRGPVIGPLVIVAAVVEEDKIRSLTKLGVKDSKQLTPAQREKLFDEIVKVLDDYSVVIVSPQDIDGRKGSMNELEVENFVKALNSLKVKPEVIYIDSADVKAERFAENIRSRLAYEAKVVAEHKADAKYEIVSAASILAKVIRDREIEKLKAEYGDFGSGYPSDPRTKKWLEEWYSKHGNFPPIVRRTWDTAKKIEEKFKRAQLTLDNFLKRFRNKLAAALEIKRA

SEQ ID NO: 3 - DNA, 43 bp, Artificial Sequence
  Synthetic oligonucleotide
ctcactcagaccagcatgattttttcatgctggtctgagtgag

SEQ ID NO: 4 - DNA, 43 bp, Artificial Sequence; identical to SEQ ID NO: 3
  Synthetic oligonucleotide

SEQ ID NO: 5 - DNA, 21 bp, Artificial Sequence
  Synthetic oligonucleotide
ctcactctaaaccccagcatt

SEQ ID NO: 6 - DNA, 26 bp, Artificial Sequence
  Synthetic oligonucleotide
ctcactctaaaccccagcattggtct

SEQ ID NO: 7 - DNA, 22 bp, Artificial Sequence
  Synthetic oligonucleotide
cagcctcatccaaaagaggaaa

SEQ ID NO: 8 - DNA, 27 bp, Artificial Sequence
  Synthetic oligonucleotide
cagcctcatccaaaagaggaaacagga

SEQ ID NO: 9 - DNA, 25 bp, Artificial Sequence
  Synthetic oligonucleotide
ctcaggaaacacagacaatgctggg

SEQ ID NO: 10 - DNA, 378 bp, Artificial Sequence
  Synthetic
aaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactac

SEQ ID NO: 11 - DNA, 20 bp, Artificial Sequence
  Synthetic oligonucleotide
aaaccttttccagtgcttca

SEQ ID NO: 12 - DNA, 20 bp, Artificial Sequence
  Synthetic oligonucleotide
ctccaggcttttcttacagt

SEQ ID NO: 13 - DNA, 20 bp, Artificial Sequence
  Synthetic oligonucleotide
ccgtgcacctctcctgccag

SEQ ID NO: 14 - DNA, 20 bp, Artificial Sequence
  Synthetic oligonucleotide
gtagtagttccttcaagctc

SEQ ID NO: 15 - DNA, 6 bp, Artificial Sequence
  Synthetic oligonucleotide - Random Hexamer
nnnnnn

SEQ ID NO: 16 - DNA, 18 bp, Artificial Sequence
  Synthetic oligonucleotide - Oligo dT
tttttttttttttttttt

SEQ ID NO: 17 - DNA, 18 bp, Artificial Sequence
  Synthetic oligonucleotide
tgtgcagaaggatggagt

SEQ ID NO: 18 - DNA, 20 bp, Artificial Sequence
  Synthetic oligonucleotide
ctggtgcttctctcaggata

SEQ ID NO: 19 - DNA, 21 bp, Artificial Sequence
  Synthetic oligonucleotide
tgtgcagaaggatggagtgga

SEQ ID NO: 20 - DNA, 25 bp, Artificial Sequence
  Synthetic oligonucleotide
tggaatatgccctgcgtaaactgga

SEQ ID NO: 21 - DNA, 27 bp, Artificial Sequence
  Synthetic oligonucleotide
tctattgttggatcatattcgtccaca

SEQ ID NO: 22 - DNA, 21 bp, Artificial Sequence
  Synthetic oligonucleotide
aacttgtggtagttggagctg

SEQ ID NO: 23 - DNA, 24 bp, Artificial Sequence
  Synthetic oligonucleotide
aacttgtggtagttggagctggtc

SEQ ID NO: 24 - DNA, 24 bp, Artificial Sequence
  Synthetic oligonucleotide
aacttgtggtagttggagctgutc

SEQ ID NO: 25 - DNA, 21 bp, Artificial Sequence
  Synthetic oligonucleotide
agagtgccttgacgatacagc

SEQ ID NO: 26 - DNA, 169 bp, Artificial Sequence
  Synthetic
tgtgcagaaggatggagtggggatggtcgagtatctcagaaaagaagacatggaatatgccctgcgtaaactggatgacaccaaattccgctctcatgagggtgaaacttcctacatccgagtttatcctgagagaagcaccagctatggctactcacggtctcggtct

SEQ ID NO: 27 - DNA, 23 bp, Artificial Sequence
  Synthetic oligonucleotide
tgtgcagaaggatggagtgggga

SEQ ID NO: 28 - DNA, 25 bp, Artificial Sequence
  Synthetic oligonucleotide
ctggtgcttctctcaggataaactc

SEQ ID NO: 29 - DNA, 20 bp, Artificial Sequence
  Synthetic oligonucleotide
agaccgagacgtgagtagcc

SEQ ID NO: 30 - DNA, 21 bp, Artificial Sequence
  Synthetic oligonucleotide
agaccgagaccgtgagtagcc

SEQ ID NO: 31 - DNA, 21 bp, Artificial Sequence
  Synthetic oligonucleotide
agaccgagaccgtgagtagcc

SEQ ID NO: 32 - DNA, 43 bp, Artificial Sequence; identical to SEQ ID NO: 3
  Synthetic oligonucleotide

SEQ ID NO: 33 - DNA, 33 bp, Artificial Sequence
  Synthetic oligonucleotide
ccagcatgattttttcatgctggtctgagtgag

SEQ ID NO: 34 - DNA, 10 bp, Artificial Sequence
  Synthetic oligonucleotide
ctcactcaga

(19) United States
(12) Patent Application Publication
(10) Pub. No.: US 2014/0255925 A9
(43) Pub. Date: Sep. 11, 2014

(54) MODIFIED RNASE H ENZYMES AND THEIR USES

(71) Applicants: Joseph Alan WALDER, Chicago, IL (US); Mark Aaron BEHLKE, Coralville, IA (US); Scott D. ROSE, Coralville, IA (US); Joseph DOBOSY, Coralville, IA (US); Susan Marie RUPP, Marion, IA (US)

(72) Inventors: Joseph Alan WALDER, Chicago, IL (US); Mark Aaron BEHLKE, Coralville, IA (US); Scott D. ROSE, Coralville, IA (US); Joseph DOBOSY, Coralville, IA (US); Susan Marie RUPP, Marion, IA (US)

(73) Assignee: Integrated DNA Technologies, Coralville, IA (US)

(21) Appl. No.: 13/839,334

(22) Filed: Mar. 15, 2013

Related U.S. Application Data
(63) Continuation-in-part of application No. 12/433,896, filed on Apr. 30, 2009, now abandoned.
Application No. 12/433,896 is a continuation-in-part of application No. 12/507,142, filed on Jul. 22, 2009.
Application No. 12/507,142 is a continuation-in-part of application No. 12/433,896, filed on Apr. 30, 2009, now abandoned.
(60) Provisional application No. 61/612,798, filed on Mar. 19, 2012.
(60) Provisional application No. 61/049,204, filed on Apr. 30, 2008.

(51) Int. Cl. C12Q 1/68

(52) U.S. Cl. CPC C12Q 1/686
USPC 435/6.11; 435/91.2; 435/199; 435/91.53

(57) ABSTRACT

The invention provides a provides improvements to assays that employ RNase H cleavage for biological applications related to nucleic acid amplification and detection, where the RNase H has been reversibly inactivated.

### CROSS REFERENCE TO RELATED APPLICATION

This application claims the benefit of priority from U.S. Provisional Application No. 61/612,798, filed Mar. 19, 2012, the disclosure of which is incorporated by reference herein in its entirety. This application additionally claims priority to U.S. application Ser. No. 12/433,896, filed Apr. 30, 2009, and U.S. application Ser. No. 12/507,142, filed Jul. 22, 2009.

The sequence listing submitted herewith is incorporated by reference in its entirety.

### FIELD OF THE INVENTION

This invention pertains to methods of chemically modifying a Ribonuclease H (RNase H) enzyme with acid anhydrides for heat-reversible inactivation, as well as applications utilizing such modified enzymes.

### BACKGROUND OF THE INVENTION

Polymerase chain reaction (PCR) is a ubiquitous method of exponentially amplifying single or small numbers of copies of DNA. Although the technique is thirty years old, its use continues to grow and is now incorporated into methods of sequencing, functional genomics, diagnostics, forensics and gene expression.

Dozens of variations of PCR now exist, and most share several basic steps: denaturation at a high temperature to break the hydrogen bonds between complementary bases and forming single-stranded target DNA; annealing of primers at a lower temperature; and extension of the primers with a thermostable polymerase enzyme that has optimum activity above 60° C., thereby amplifying the target DNA. PCR is not a perfect system, and typically non-specific amplification occurs at lower temperatures where the thermostable enzymes still have slight activity. Many applications of PCR are hindered by this limitation, and “Hot start PCR” methods have been devised to reduce or eliminate non-specific amplification.

“Hot start PCR” refers to the use of methods that prevent initiation of the polymerase chain reaction until the reaction has been heated to a high temperature, usually at or around 95° C., and cooled to the primer annealing temperature, usually at or around 60° C. The first hot start PCR methods employed physical barriers that could be disrupted by heating to remove the barriers between the reaction components. In one of the early embodiments of this approach, the nucleic acids (target DNA, primers, deoxynucleotides, and buffer) are separated from the DNA polymerase by a wax seal. The wax melts when the reaction is heated to 95° C., permitting mixing of the DNA polymerase with the other reaction components, and PCR commences once the reaction cools sufficiently for primer binding to occur. Today, hot start PCR is usually performed using a homogenous reaction mix wherein the DNA polymerase is inactivated by some method that can be reversed by heating. Examples include chemical modification (such as the anhydride modification schemes used in the present invention to reversibly inactivate RNase H2), antibodies that bind the DNA polymerase, or aptamers that bind the DNA polymerase. In all cases, the agent limiting DNA polymerase activity is reversed, denatured, or degraded by heating.

A reversibly-inactivated hot-start Taq DNA polymerase typically costs 5-10 fold more than unmodified native Taq polymerase. In spite of increased cost, hot start PCR is almost exclusively used in PCR applications today. Use of hot start methods improves the outcome of PCR in two ways:
1) Increased specificity. In the absence of hot start methods, primers can bind at low temperatures at sites in a complex nucleic acid sample having an imperfect sequence match and initiate DNA synthesis, leading to amplification of undesired products. Hot start methods can reduce or prevent mis-priming of this type.
2) Permits reactions to remain inert at room temperature before PCR cycling is begun. It is common for high-throughput screening methods to involve set-up of dozens of PCR plates (comprising thousands of individual reactions) which are held at room temperature for later loading into a PCR thermocycler by robotic lab instrumentation. In the absence of hot start methods, side reactions occur that are dependent on the DNA polymerase and primers which consume reagents and compromise the quality of PCR once it finally commences.

Variations of PCR have been developed that utilize other enzymes that are inherently inactive at lower temperatures, thereby limiting undesired non-specific amplification. One example, described by Walder et al., (U.S. Patent Application 2009/0325169), uses a primer containing a blocking group at or near the 3′-end. The primer cannot extend until the blocking group is cleaved by an RNase H enzyme that has little to no activity at lower temperatures.

RNase H is an endoribonuclease that cleaves the phosphodiester bond in an RNA strand when it is part of an RNA:DNA duplex. The enzyme does not cleave DNA or unhybridized single-stranded RNA. This characteristic makes RNase H useful in biological applications, such as in cDNA synthesis wherein the RNA template is destroyed once the desired complementary DNA is synthesized by reverse transcription.

Anhydride modifications have been extensively used to modify proteins for heat-reversible inactivation, most commonly when applied to thermostable DNA polymerases such as the common DNA polymerase from Thermus aquaticus (Taq) (See Birch et al. U.S. Pat. No. 5,773,258). These anhydrides take the general structure as shown in Formula I wherein R₁ and R₂ are hydrogen or substituted or unsubstituted alkyl or aryl groups, or R₁ and R₂ form a cyclic group.

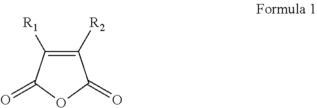

Examples of the preferred anhydrides include but are not limited to citraconic anhydride and 3,4,5,6-Tetrahydrophthalic anhydride. These reagents were reacted with the RNase H2 protein to generate the reversible inactivation. The anhydrides modify the terminal amines of lysines and the N-terminus of the protein, altering the charge and likely affecting the conformation of the protein (FIG. 1). These protein modifications are known to be highly sensitive to high temperature and low pH (see Dixon and Perham, Biochem J 1968, 109(2):312-314), with different removal kinetics dependent on the nature of the anhydride utilized (see Walder et al., Mol Pharmacol 1977, 13(3):407-414).

The current invention also provides improvements to assays that employ RNase H cleavage for biological applications related to nucleic acid amplification and detection, where the RNase H has been reversibly inactivated. These and other advantages of the invention, as well as additional inventive features, will be apparent from the description of the invention provided herein.

### BRIEF SUMMARY OF THE INVENTION

The invention provides a provides improvements to assays that employ RNase H cleavage for biological applications related to nucleic acid amplification and detection, where the RNase H has been reversibly inactivated.

The utility of RNase H, particularly thermophilic RNase H enzymes, also extends to a number of other biological assays (see Walder et al., U.S. Application Number 2009/0325169, incorporated herein in its entirety). Thermophilic RNase H enzymes can enable hot start protocols in nucleic acid amplification and detection assays including but not limited to PCR, OLA (oligonucleotide ligation assays), LCR (ligation chain reaction), polynomial amplification and DNA sequencing, wherein the hot start component is a thermostable RNase H or other nicking enzyme that gains activity at the elevated temperatures employed in the reaction. Such assays employ a modified oligonucleotide of the invention that is unable to participate in the reaction until it hybridizes to a complementary nucleic acid sequence and is cleaved to generate a functional 5′- or 3′-end. Compared to the corresponding assays in which standard unmodified DNA oligonucleotides are used, the specificity is greatly enhanced. Moreover the requirement for reversibly inactivated DNA polymerases or DNA ligases is eliminated.

There are several alternatives for hot start RNase H: 1) a thermostable RNase H enzyme that has intrinsically little or no activity at reduced temperatures as in the case of Pyrococcus abysii RNase H2; 2) a thermostable RNase H reversibly inactivated by chemical modification; and 3) a thermostable RNase H reversibly inactivated by a blocking antibody. In addition, through means well-known in the art, such as random mutagenesis, mutant versions of RNase H can be synthesized that can further improve the traits of RNase H that are desirable in the assays of the present invention. Alternatively, mutant strains of other enzymes that share the characteristics desirable for the present invention could be used. The methods of the present invention are primarily directed to the second alternative.

### BRIEF DESCRIPTION OF THE DRAWINGS

FIG. 1 contains structures of various anhydride compounds useful for protein modification. The chemical structures of cis-aconitic anhydride, citraconic anhydride, and 3,4,5,6-tetrahydrophthalic anhydride are shown.

FIG. 2 shows a scheme for reaction of a lysine residue with 3,4,5,6-tetrahydrophthalic anhydride and removal of the anhydride by heat treatment. The reaction scheme for coupling 3,4,5,6-tetrahydrophthalic anhydride to a lysine residue is shown (top), which results in inactivation of a modified enzyme. Treatment of this structure with heat or low pH reverses the reaction (bottom), which results in re-activation of the now unmodified enzyme.

FIG. 3 shows a FQ-Reporter oligonucleotide assay for RNase H2 activity. A fluorescence-quenched hairpin probe assay for RNase H2 activity is shown. DNA bases are uppercase, RNA bases are lower case, FAM is 6-carboxyfluorescein, and FQ is Iowa Black® FQ dark quencher. In the intact state, the probe forms a hairpin which aligns the FAM reporter dye with the Iowa Black dark quencher. In this configuration, the probe is “dark”. Cleavage of the probe by RNase H2 occurs at the 5′-side of the ribo-C residue. At the elevated reaction temperatures, the cleaved fragment dissociates, separating the reporter dye from the quencher. In this state the probe is “bright” and a positive signal is detected at 520 nm FAM emission.

FIG. 4 shows an assay of inactivation and heat reactivation of 3,4,5,6-tetrahydrophthalic anhydride-modified P.a. RNase H2 using 2.6 mU enzyme. The relative activity of unmodified and 3,4,5,6-tetrahydrophthalic anhydride-modified P.a. RNase H2 was characterized using the FQ-reporter oligonucleotide assay. Assays were run in 10 μL reactions using 0.9 nM enzyme (2.6 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 5 shows an assay of inactivation and heat reactivation of 3,4,5,6-tetrahydrophthalic anhydride-modified P.a. RNase H2 using 200 mU enzyme. The relative activity of unmodified and 3,4,5,6-tetrahydrophthalic anhydride-modified P.a. RNase H2 was characterized using the FQ-reporter oligonucleotide assay. Assays were run in 10 μL reactions using 67 nM enzyme (200 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 6 shows an assay of inactivation and heat reactivation of cis-aconitic anhydride-modified P.a. RNase H2 using 2.6 mU enzyme. The relative activity of unmodified and cis-aconitic anhydride-modified P.a. RNase H2 was characterized using the FQ-reporter oligonucleotide assay. Assays were run in 10 μL reactions using 0.9 nM enzyme (2.6 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 7 shows an assay of inactivation and heat reactivation of cis-aconitic anhydride-modified P.a. RNase H2 using 200 mU enzyme. The relative activity of unmodified and cis-aconitic anhydride-modified P.a. RNase H2 was characterized using the FQ-reporter oligonucleotide assay. Assays were run in 10 μL reactions using 67 nM enzyme (200 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 8 shows an assay of inactivation and heat reactivation of citraconic anhydride-modified P.a. RNase H2 using 2.6 mU enzyme. The relative activity of unmodified and citraconic anhydride-modified P.a. RNase H2 was characterized using the FQ-reporter oligonucleotide assay. Assays were run in 10 μL reactions using 0.9 nM enzyme (2.6 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 9 shows an assay of inactivation and heat reactivation of citraconic anhydride-modified P.a. RNase H2 using 200 mU enzyme. The relative activity of unmodified and citraconic anhydride-modified P.a. RNase H2 was characterized using the FQ-reporter oligonucleotide assay. Assays were run in 10 μL reactions using 67 nM enzyme (200 mU for the unmodified enzyme) at 60° C. Fluorescence measurements were collected every 11 seconds during the 10 minute incubation. Enzyme was either added directly to the reactions (top panel) or following 10 minutes incubation at 95° C. to reverse the anhydride modification and reactivate enzyme activity (bottom panel). RFUs are relative fluorescence units.

FIG. 10 shows the ESI-MS spectra of unmodified recombinant Pyrococcus abyssi RNase H2. P.a. RNase H2 was examined by electrospray ionization mass spectrometry (ESI-MS). A deconvolution trace of the mass spectra is shown and the molecular weight (Daltons, Da) of the primary peak is indicated.

FIG. 11 shows ESI-MS spectra of recombinant Pyrococcus abyssi RNase H2 modified with 3,4,5,6-tetrahydrophthalic anhydride. P.a. RNase H2 was reacted with a total of 3-fold molar excess of 3,4,5,6-tetrahydrophthalic anhydride and the modified protein was examined by electrospray ionization mass spectrometry (ESI-MS). A deconvolution trace of the mass spectra is shown and the molecular weights (Daltons) of the primary peaks are indicated.

FIG. 12 shows ESI-MS spectra of recombinant Pyrococcus abyssi RNase H2 modified with 3,4,5,6-tetrahydrophthalic anhydride followed by heat treatment. P.a. RNase H2 was reacted with a total of 3-fold molar excess of 3,4,5,6-tetrahydrophthalic anhydride and the modified protein was heated at 95° C. for 10 minutes to reverse the modification reaction. The final product was examined by electrospray ionization mass spectrometry (ESI-MS). A deconvolution trace of the mass spectra is shown and the molecular weights (Daltons) of the primary peaks are indicated.

FIG. 13 shows amplification plots of qPCR done after overnight incubation at room temperature using a hot-start DNA polymerase. Amplification reactions were performed using a hot start DNA polymerase (iTaq). All reaction components were mixed together and reaction plates were incubated overnight at room temperature. Use of unmodified primers (left panels) resulted in efficient amplification reactions and no difference was seen between addition of native P.a. RNase H2 (top left) and 3,4,5,6-tetrahydrophthalic anhydride-modified hot start P.a. RNase H2 (bottom left). Use of blocked-cleavable primers (right panels) resulted in efficient amplification reactions and no difference was seen between addition of native P.a. RNase H2 (top right) and 3,4,5,6-tetrahydrophthalic anhydride-modified hot start P.a. RNase H2 (bottom right).

FIG. 14 shows amplification plots of qPCR done after overnight incubation at room temperature using native (non-hot start) Taq DNA polymerase. Amplification reactions were performed using native Taq DNA polymerase (not hot start). All reaction components were mixed together and reaction plates were incubated overnight at room temperature. Use of unmodified primers (left panels) resulted in no detectable amplification of the target nucleic acid sequence; reactions were run with native P.a. RNase H2 (top left) and 3,4,5,6-tetrahydrophthalic anhydride-modified hot start P.a. RNase H2 (bottom left). Use of blocked-cleavable primers (right panels) resulted in efficient amplification of the target nucleic acid when 3,4,5,6-tetrahydrophthalic anhydride-modified hot start P.a. RNase H2 was employed (bottom right) but not when native P.a. RNase H2 was employed (top right).

FIG. 15 contains amplification plots of RT-qPCR detecting the human SFRS9 gene using high temperature RT with unmodified primers and native P.a. RNase H2. Reactions were performed using the HawkZ05™ Fast One-Step RT-PCR Master mix in a single-tube format with unmodified Forward and Reverse PCR primers. The Reverse PCR primer also functioned as the RT primer. The reverse transcription (RT) reaction was done using 20 ng of HeLa cell RNA and proceeded in a stepwise fashion with incubations of 5 minutes at 55° C., 5 minutes at 60° C., and 5 minutes at 65° C. followed by a 10 minute denaturation step at 95° C., after which 45 cycles of PCR was performed. Reactions were done without RNase H2 or with 2.6 mU, 25 mU, or 200 mU of native P.a. RNase H2 as indicated.

FIG. 16 contains amplification plots of RT-qPCR detecting the human SFRS9 gene using high temperature RT with unmodified primers and anhydride-modified HS-P.a. RNase H2. Reactions were performed using the HawkZ05™ Fast One-Step RT-PCR Master mix in a single-tube format with unmodified Forward and Reverse PCR primers. The Reverse PCR primer also functioned as the RT primer. The reverse transcription (RT) reaction was done using 20 ng of HeLa cell RNA and proceeded in a stepwise fashion with incubations of 5 minutes at 55° C., 5 minutes at 60° C., and 5 minutes at 65° C. followed by a 10 minute denaturation/RNase H2 activation step at 95° C., after which 45 cycles of PCR was performed. Reactions were done without RNase H2 or with 2.6 mU, 25 mU, or 200 mU of 3,4,5,6-tetrahydrophthalic anhydride—modified P.a. RNase H2 as indicated.

FIG. 17 contains amplification plots of RT-qPCR detecting the human SFRS9 gene using high temperature RT with a blocked-cleavable For PCR primer and anhydride-modified HS-P.a. RNase H2. Reactions were performed using the HawkZ05™ Fast One-Step RT-PCR Master mix in a single-tube format with a blocked-cleavable Forward PCR primer and an unmodified Reverse PCR primer. The Reverse PCR primer also functioned as the RT primer. The reverse transcription (RT) reaction was done using 20 ng of HeLa cell RNA and proceeded in a stepwise fashion with incubations of 5 minutes at 55° C., 5 minutes at 60° C., and 5 minutes at 65° C. followed by a 10 minute denaturation/RNase H2 activation step at 95° C., after which 45 cycles of PCR was performed. Reactions were done without RNase H2 or with 2.6 mU, 25 mU, or 200 mU of 3,4,5,6-tetrahydrophthalic anhydride—modified P.a. RNase H2 as indicated.

FIG. 18 shows amplification products of the SFRS9 gene from high-temperature RT-qPCR using 3,4,5,6-tetrahydrophthalic anhydride—modified P.a. RNase H2 and an unmodified external RT primer. Reactions were performed using the HawkZ05™ Fast One-Step RT-PCR Master mix in a single-tube format with unmodified (U) For and Rev PCR primers or blocked-cleavable (B) For and Rev rhPCR primers. The reverse transcription (RT) reaction was done using 10 ng of HeLa cell RNA and proceeded in a stepwise fashion with incubations of 5 minutes at 55° C., 5 minutes at 60° C., and 5 minutes at 65° C. followed by a 10 minute denaturation/RNase H2 activation step at 95° C., after which 45 cycles of PCR was performed. Reactions were done with 10 mU of 3,4,5,6-tetrahydrophthalic anhydride—modified P.a. RNase H2. An unmodified external RT primer was employed at the concentrations indicated. Position of the desired 145 bp amplicon is indicated (made from the For and Rev PCR primers). Position of the undesired 170 bp amplicon is indicated (made from the For PCR primer and the RT primer).

FIG. 19 shows amplification products of the SFRS9 gene from high-temperature RT-qPCR using 3,4,5,6-tetrahydrophthalic anhydride—modified P.a. RNase H2 and a modified external RT primer containing a central rC RNA residue. Reactions were performed using the HawkZ05™ Fast One-Step RT-PCR Master mix in a single-tube format with unmodified (U) For and Rev PCR primers or blocked-cleavable (B) For and Rev rhPCR primers. The reverse transcription (RT) reaction was done using 10 ng of HeLa cell RNA and proceeded in a stepwise fashion with incubations of 5 minutes at 55° C., 5 minutes at 60° C., and 5 minutes at 65° C. followed by a 10 minute denaturation/RNase H2 activation step at 95° C., after which 45 cycles of PCR was performed. Reactions were done with 10 mU of 3,4,5,6-tetrahydrophthalic anhydride—modified P.a. RNase H2. A modified external RT primer containing a single centrally-positioned rC RNA residue was employed at the concentrations indicated. Position of the desired 145 bp amplicon is indicated (made from the For and Rev PCR primers). Position of the undesired 170 bp amplicon is indicated (made from the For PCR primer and the RT primer). Control reactions were run in the absence of any external RT primer (0 nM).

FIG. 20 shows amplification products of the SFRS9 gene from high-temperature RT-qPCR using 3,4,5,6-tetrahydrophthalic anhydride—modified P.a. RNase H2 and a modified external RT primer containing a central abasic napthyl-azo modifier. Reactions were performed using the HawkZ05™ Fast One-Step RT-PCR Master mix in a single-tube format with unmodified (U) For and Rev PCR primers or blocked-cleavable (B) For and Rev rhPCR primers. The reverse transcription (RT) reaction was done using 10 ng of HeLa cell RNA and proceeded in a stepwise fashion with incubations of 5 minutes at 55° C., 5 minutes at 60° C., and 5 minutes at 65° C. followed by a 10 minute denaturation/RNase H2 activation step at 95° C., after which 45 cycles of PCR was performed. Reactions were done with 10 mU of 3,4,5,6-tetrahydrophthalic anhydride—modified P.a. RNase H2. A modified external RT primer containing a single centrally-positioned abasic napthyl-azo modifier was employed at the concentrations indicated. Position of the desired 145 bp amplicon is indicated (made from the For and Rev PCR primers). Position of the undesired 170 bp amplicon is indicated (made from the For PCR primer and the RT primer).

### DETAILED DESCRIPTION OF THE INVENTION

In one embodiment, the compositions and methods of the invention involve modification of an RNase H2 enzyme to make it reversibly inactivated, and become reactivated upon heating. RNase H2 is modified with acid anhydrides to generate a chemically modified hot-start RNase H2 enzyme (HS-RNase H2). In a further embodiment, the RNase H2 enzyme is from the organism Pyrococcus abyssi (P.a.). The methodologies described in this disclosure also describe the improved utility of the HS-RNase H2 in PCR and reverse-transcription PCR (RT-PCR) assays.

The use of blocked-cleavable primers with RNase H2 increases the specificity of PCR (rhPCR). Further, DNA synthesis reactions that are dependent on primers cannot occur using blocked-cleavable primers until the primers have been activated by RNase H2 cleavage; certain RNase H2 enzymes, such as P.a. RNase H2, have minimal activity at room temperature. It is therefore possible that rhPCR may perform well using native Taq DNA polymerase, avoiding the need for a costly commercial hot start DNA polymerase; i.e., rhPCR may inherently display hot-start behavior. Throughout the application, unless otherwise stated, references to HS-RNase H2 refer to non-native RNase H2.

In one embodiment, the HS-RNase H2 also can be used in high temperature RT reactions. It may be beneficial to use high-specificity rhPCR (which employs blocked-cleavable primers and RNase H2) to quantify target gene levels in cDNA, which is made from RNA by reverse transcription (RT). RT reactions employ DNA oligonucleotides to prime synthesis of cDNA from an RNA template. The priming complex forms an RNA:DNA heteroduplex, so the presence of RNase H2 activity in an RT reaction could degrade the target RNA, decreasing the efficiency of the reaction. RT-qPCR is often done as a 2-step process, where the RT reaction is first done at low temperature (typically 37-42° C.), for example using the avian myeloblastosis virus (AMV) RT enzyme or the Moloney murine leukemia virus (MMLV) RT enzyme. Following completion of the cDNA synthesis reaction, PCR is performed at high temperature (typically 60-72° C.). If these reactions are performed in separate tubes, the RNase H2 enzyme can be added after cDNA synthesis is complete. If RT and PCR steps are linked in a single tube, then the RNase H2 must be present during RT and may degrade the RNA target. Example 5 demonstrates an additional advantage of the invention, whereby SNPs can be identified in RNA sequences using the HS-RNase H2 and rhPCR. This can be used in many diverse fields, anywhere that RNA must be analyzed for sequence changes.

The ability of one-tube RT-PCR to be performed with blocked primers and with the HS-RNase H2 enzyme is demonstrated in Example 4, where a single blocked primer is employed with an unblocked reverse primer which acts as both the RT primer and as the reverse primer in the subsequent PCR.

The ability of the HS-RNase H2 to perform in RT-qPCR single-nucleotide polymorphism (SNP) assays is demonstrated in Example 5, where a single nucleotide difference between two different RNA samples is detected using a one-tube RT-PCR system and a single blocked primer with the potential SNP placed opposite the RNA base.

The ability of the HS-RNase H2 to perform in RT-qPCR assays containing two blocked primers and an external unblocked reverse transcription primer is demonstrated in Example 11 below.

The HS-RNase H2 also can be used in high temperature RT reactions, where the activity of the native enzyme would destroy the RNA before it could be reverse-transcribed. This advantage is displayed in examples 9 and 10. Example 9 demonstrates and additional advantage of the invention, whereby SNPs can be identified in RNA sequences using the HS-RNase H2 and rhPCR. This can be used in many diverse fields, anywhere that RNA must be analyzed for sequence changes.

The P.a RNase H2 has low activity at 25° C., but this may not be sufficient when long pre-incubation times occur before the rhPCR is performed (i.e. when large numbers of reactions are performed in batch with a robot). The HS-RNase H2 allows for the reversible inactivation of the enzyme to occur, and allows for complete return to functionality when required. An example of this advantage is shown in example 11.

### DEFINITIONS

To aid in understanding the invention, several terms are defined below.

The terms “nucleic acid” and “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, N.Y.; Wetmur, 1991, Critical Review in Biochem. and Mol. Biol. 26(3/4):227-259; and Owczarzy et al., 2008, Biochemistry, 47: 5336-5353, which are incorporated herein by reference).

The terms “target, “target sequence”, “target region”, and “target nucleic acid,” as used herein, are synonymous and refer to a region or sequence of a nucleic acid which is to be amplified, sequenced or detected.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (e.g., a DNA polymerase or reverse transcriptase) in an appropriate buffer and at a suitable temperature. Primer extension can also be carried out in the absence of one or more of the nucleotide triphosphates in which case an extension product of limited length is produced. As used herein, the term “primer” is intended to encompass the oligonucleotides used in ligation-mediated reactions, in which one oligonucleotide is “extended” by ligation to a second oligonucleotide which hybridizes at an adjacent position. Thus, the term “primer extension”, as used herein, refers to both the polymerization of individual nucleoside triphosphates using the primer as a point of initiation of DNA synthesis and to the ligation of two oligonucleotides to form an extended product.

A primer is preferably a single-stranded DNA. The appropriate length of a primer depends on the intended use of the primer but typically ranges from 6 to 50 nucleotides, preferably from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product. The region of the primer which is sufficiently complementary to the template to hybridize is referred to herein as the hybridizing region.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two step cycles have a high temperature denaturation step followed by a hybridization/elongation (or ligation) step. Three step cycles comprise a denaturation step followed by a hybridization step followed by a separate elongation or ligation step.

As used herein, a “polymerase” refers to an enzyme that catalyzes the polymerization of nucleotides. Generally, the enzyme will initiate synthesis at the 3′-end of the primer annealed to a nucleic acid template sequence. “DNA polymerase” catalyzes the polymerization of deoxyribonucleotides. Known DNA polymerases include, for example, Pyrococcus furiosus (Pfu) DNA polymerase (Lundberg et al., 1991, Gene, 108:1), E. coli DNA polymerase I (Lecomte and Doubleday, 1983, Nucleic Acids Res. 11:7505), T7 DNA polymerase (Nordstrom et al., 1981, J. Biol. Chem. 256:3112), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Nucleic Acids Res, 19: 4193), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), Pyrococcus kodakaraensis KOD DNA polymerase (Takagi et al., 1997, Appl. Environ. Microbiol. 63:4504), JDF-3 DNA polymerase (Patent application WO 0132887), and Pyrococcus GB-D (PGB-D) DNA polymerase (Juncosa-Ginesta et al., 1994, Biotechniques, 16:820). The polymerase activity of any of the above enzymes can be determined by means well known in the art.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences which contain the target primer binding sites.

The term “non-specific amplification,” as used herein, refers to the amplification of nucleic acid sequences other than the target sequence which results from primers hybridizing to sequences other than the target sequence and then serving as a substrate for primer extension. The hybridization of a primer to a non-target sequence is referred to as “non-specific hybridization” and is apt to occur especially during the lower temperature, reduced stringency, pre-amplification conditions, or in situations where there is a variant allele in the sample having a very closely related sequence to the true target as in the case of a single nucleotide polymorphism (SNP).

The term “reaction mixture,” as used herein, refers to a solution containing reagents necessary to carry out a given reaction. An “amplification reaction mixture”, which refers to a solution containing reagents necessary to carry out an amplification reaction, typically contains oligonucleotide primers and a DNA polymerase or ligase in a suitable buffer. A “PCR reaction mixture” typically contains oligonucleotide primers, a DNA polymerase (most typically a thermostable DNA polymerase), dNTP's, and a divalent metal cation in a suitable buffer. A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components which includes the blocked primers of the invention.

The term “cleavage domain” or “cleaving domain,” as used herein, are synonymous and refer to a region located between the 5′ and 3′ end of a primer or other oligonucleotide that is recognized by a cleavage compound, for example a cleavage enzyme, that will cleave the primer or other oligonucleotide. For the purposes of this invention, the cleavage domain is designed such that the primer or other oligonucleotide is cleaved only when it is hybridized to a complementary nucleic acid sequence, but will not be cleaved when it is single-stranded. The cleavage domain or sequences flanking it may include a moiety that a) prevents or inhibits the extension or ligation of a primer or other oligonucleotide by a polymerase or a ligase, b) enhances discrimination to detect variant alleles, or c) suppresses undesired cleavage reactions. One or more such moieties may be included in the cleavage domain or the sequences flanking it.

The term “RNase H cleavage domain,” as used herein, is a type of cleavage domain that contains one or more ribonucleic acid residue or an alternative analog which provides a substrate for an RNase H. An RNase H cleavage domain can be located anywhere within a primer or oligonucleotide, and is preferably located at or near the 3′-end or the 5′-end of the molecule.

An “RNase H1 cleavage domain” generally contains at least three residues. An “RNase H2 cleavage domain” may contain one RNA residue, a sequence of contiguously linked RNA residues or RNA residues separated by DNA residues or other chemical groups. In one embodiment, the RNase H2 cleavage domain is a 2′-fluoronucleoside residue. In a more preferred embodiment the RNase H2 cleavable domain is two adjacent 2′-fluoro residues.

The terms “cleavage compound,” or “cleaving agent” as used herein, refers to any compound that can recognize a cleavage domain within a primer or other oligonucleotide, and selectively cleave the oligonucleotide based on the presence of the cleavage domain. The cleavage compounds utilized in the invention selectively cleave the primer or other oligonucleotide comprising the cleavage domain only when it is hybridized to a substantially complementary nucleic acid sequence, but will not cleave the primer or other oligonucleotide when it is single stranded. The cleavage compound cleaves the primer or other oligonucleotide within or adjacent to the cleavage domain. The term “adjacent,” as used herein, means that the cleavage compound cleaves the primer or other oligonucleotide at either the 5′-end or the 3′ end of the cleavage domain. Cleavage reactions preferred in the invention yield a 5′-phosphate group and a 3′-OH group.

In a preferred embodiment, the cleavage compound is a “cleaving enzyme.” A cleaving enzyme is a protein or a ribozyme that is capable of recognizing the cleaving domain when a primer or other nucleotide is hybridized to a substantially complementary nucleic acid sequence, but that will not cleave the complementary nucleic acid sequence (i.e., it provides a single strand break in the duplex). The cleaving enzyme will also not cleave the primer or other oligonucleotide comprising the cleavage domain when it is single stranded. Examples of cleaving enzymes are RNase H enzymes and other nicking enzymes.

The term “blocking group,” as used herein, refers to a chemical moiety that is bound to the primer or other oligonucleotide such that an amplification reaction does not occur. For example, primer extension and/or DNA ligation does not occur. Once the blocking group is removed from the primer or other oligonucleotide, the oligonucleotide is capable of participating in the assay for which it was designed (PCR, ligation, sequencing, etc). Thus, the “blocking group” can be any chemical moiety that inhibits recognition by a polymerase or DNA ligase. The blocking group may be incorporated into the cleavage domain but is generally located on either the 5′- or 3′-side of the cleavage domain. The blocking group can be comprised of more than one chemical moiety. In the present invention the “blocking group” is typically removed after hybridization of the oligonucleotide to its target sequence.

The term “fluorescent generation probe” refers either to a) an oligonucleotide having an attached fluorophore and quencher, and optionally a minor groove binder or to b) a DNA binding reagent such as SYBR® Green dye.

The terms “fluorescent label” or “fluorophore” refers to compounds with a fluorescent emission maximum between about 350 and 900 nm. A wide variety of fluorophores can be used, including but not limited to: 5-FAM (also called 5-carboxyfluorescein; also called Spiro(isobenzofuran-1(3H), 9′-(9H)xanthene)-5-carboxylic acid, 3′,6′-dihydroxy-3-oxo-6-carboxyfluorescein); 5-Hexachloro-Fluorescein; ([4,7,2′,4′,5′,7′-hexachloro-(3′,6′-dipivaloyl-fluoresceinyl)-6-carboxylic acid]); 6-Hexachloro-Fluorescein; ([4,7,2′,4′,5′,7′-hexachloro-(3′,6′-dipivaloylfluoresceinyl)-5-carboxylic acid]); 5-Tetrachloro-Fluorescein; ([4,7,2′,7′-tetra-chloro-(3′,6′-dipivaloylfluoresceinyl)-5-carboxylic acid]); 6-Tetrachloro-Fluorescein; ([4,7,2′,7′-tetrachloro-(3′,6′-dipivaloylfluoresceinyl)-6-carboxylic acid]); 5-TAMRA (5-carboxytetramethylrhodamine); Xanthylium, 9-(2,4-dicarboxyphenyl)-3,6-bis(dimethyl-amino); 6-TAMRA (6-carboxytetramethylrhodamine); 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); Cy5 (Indodicarbocyanine-5); Cy3 (Indo-dicarbocyanine-3); and BODIPY FL (2,6-dibromo-4,4-difluoro-5,7-dimethyl-4-bora-3a,4a-diaza-s-indacene-3-proprionic acid); Quasar®-670 dye (Biosearch Technologies); Cal Fluor® Orange dye (Biosearch Technologies); Rox dyes; Max dyes (Integrated DNA Technologies), as well as suitable derivatives thereof.

As used herein, the term “quencher” refers to a molecule or part of a compound, which is capable of reducing the emission from a fluorescent donor when attached to or in proximity to the donor. Quenching may occur by any of several mechanisms including fluorescence resonance energy transfer, photo-induced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more. A number of commercially available quenchers are known in the art, and include but are not limited to DABCYL, Black Hole™ Quenchers (BHQ-1, BHQ-2, and BHQ-3), Iowa Black® FQ and Iowa Black® RQ. These are so-called dark quenchers. They have no native fluorescence, virtually eliminating background problems seen with other quenchers such as TAMRA which is intrinsically fluorescent.

The following examples further illustrate the invention but, of course, should not be construed as in any way limiting its scope.

### Example 1

This example demonstrates reversible modification/inactivation of Pyrococcus abysii (P.a.) RNase H2 by reaction with various anhydrides.

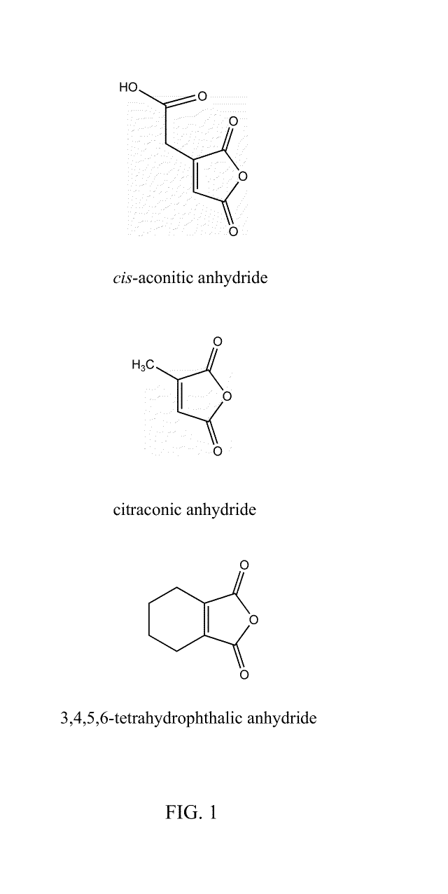

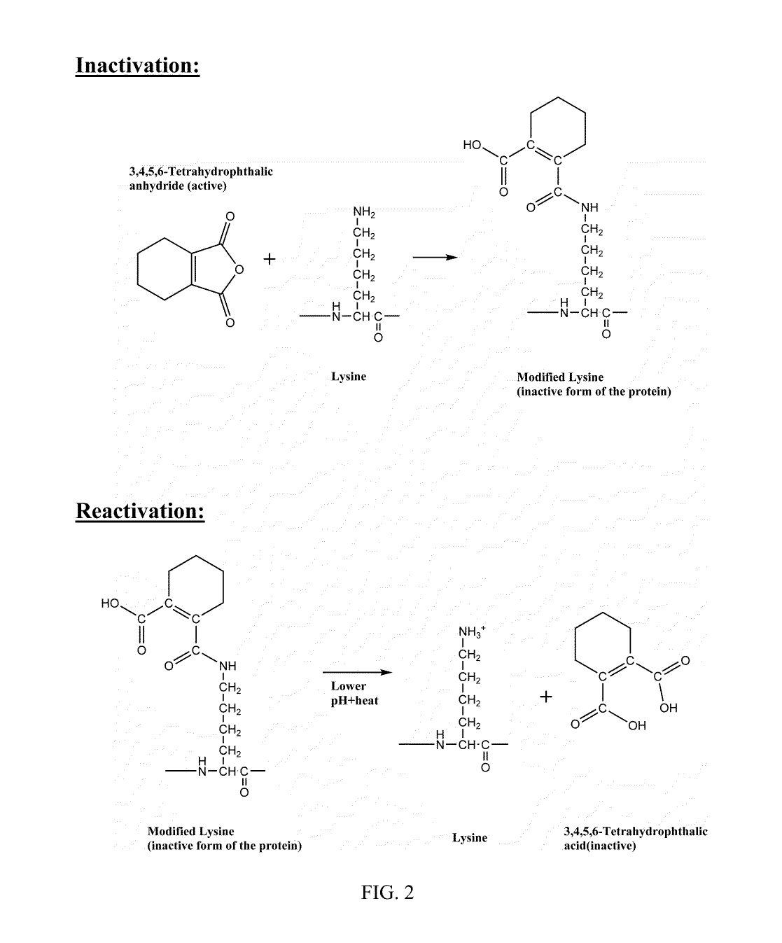

P.a. RNase H2 was chemically modified using 3,4,5,6-tetrahydrophthalic anhydride, citraconic anhydride, or cis-aconitic anhydride, and the chemical modification was removed by heat treatment. Chemical structures of the three anhydrides employed in this study are shown in FIG. 1. The chemical reaction that occurs where 3,4,5,6-tetrahydrophthalic anhydride modifies a primary amine on a polypeptide is shown in FIG. 2 as well as the reverse reaction that is catalyzed by heat. The relative enzymatic activity of the unmodified enzyme was compared with the chemically-modified enzyme before and after heat treatment.

Methods: The P.a. rnb gene was codon optimized for expression in E. coli and cloned into an expression vector as previously described (Dobosy et al., BMC Biotechnology 2011, 11:e80; Walder et al., US 2009/0325169A1). E. coli bearing the recombinant P.a. RN2 expression plasmid was grown in a 10 L fermentation reactor by the University of Iowa Center for Biocatalysis and Bioprocessing (Coralville, Iowa, USA). The resulting cell paste was stored at −80° C. A fraction of the cell paste (˜50 grams) was lysed and the recombinant P.a. RNase H2 enzyme was purified to near homogeneity by Enzymatics (Beverly, Mass., USA). Stock solutions of the enzyme were stored in Buffer F (20 mM Tris pH 8.4, 0.1 mM EDTA, 100 mM KCl, 0.1% Triton X-100, and 50% glycerol) at −20° C.

The sequence of wild-type P.a. RNase H2 is shown as SEQ ID No. 1 below. Lysine residues (K) are indicated in bold and are underscored.

SEQ ID NO. 1: Native P.a. RNase H2,
224 amino acids, 25394.18 Daltons
MKVAGADEAGRGPVIGPLVIVAAVVEEDKIRSLTKLGVKDSKQLTPAQR
EKLFDEIVKVLDDYSVVIVSPQDIDGRKGSMNELEVENFVKALNSLKVK
PEVIYIDSADVKAERFAENIRSRLAYEAKVVAEHKADAKYEIVSAASIL
AKVIRDREIEKLKAEYGDFGSGYPSDPRTKKWLEEWYSKHGNFPPIVRR
TWDTAKKIEEKFKRAQLTLDNFLKRFRN

The recombinant protein includes some additional amino acids introduced by the expression vector, which are separated from the native enzyme by a vertical bar (I). The sequence of the recombinant P.a. RNase H2 enzyme is shown below.

SEQ ID NO. 2: Recombinant P.a. RNase H2,
246 amino acids, 27573.70 Daltons
AMDIGINSDP|MKVAGADEAGRGPVIGPLVIVAAVVEEDKIRSLTKLGV
KDSKQLTPAQREKLFDEIVKVLDDYSVVIVSPQDIDGRKGSMNELEVEN
FVKALNSLKVKPEVIYIDSADVKAERFAENIRSRLAYEAKVVAEHKADA
KYEIVSAASILAKVIRDREIEKLKAEYGDFGSGYPSDPRTKKWLEEWYS
KHGNFPPIVRRTWDTAKKIEEKFKRAQLTLDNFLKRFRN|KLAAALEIK
RA

The recombinant P.a. RNase H2 protein contains 28 lysine residues. Including the amino-terminus, a total of 29 free amine groups are therefore available to be modified by chemical reaction with any of the 3 anhydrides. For chemical treatment of P.a. RNase H2 with an anhydride, a molar ratio of 29:1 of anhydride: protein therefore represents a 1:1 ratio of anhydride to total reactive amines. For simplicity, henceforth a “1:1 treatment” of P.a. RNase H2 with an anhydride will indicate use of a molar ratio of 29:1 of anhydride: protein, indicating that sufficient reagent was employed to react with every free amine group, assuming 100% efficiency.

Modification of P.a. RNase H2 with 3,4,5,6-tetrahydrophthalic anhydride, citraconic anhydride, or cis-aconitic anhydride. Three identical solutions of P.a. RNase H2 were made by adding 77 μg (864 units) of the concentrated stock recombinant enzyme into 74 μL of a buffer comprising 150 mM NaBorate (pH 9.0) and 0.1% Triton X-100, resulting in a final concentration of 38 μM. Note that reactions were performed in borate buffer, avoiding Tris-containing solutions, since the anhydrides can react with the free amine in Tris, quenching the reaction. Fresh 3,4,5,6-tetrahydrophthalic anhydride, citraconic anhydride, or cis-aconitic anhydride were dissolved in DMF at 40 mM. 1.0 μL of the 3,4,5,6-tetrahydrophthalic anhydride was added to the first RNase H2 aliquot, 1.0 μL of the citraconic anhydride was added to the second RNase H2 aliquot, and 1.0 μL of the cis-aconitic anhydride was added to the third RNase H2 aliquot. These treatments represent addition of 14.5:1 anhydride to enzyme, or a 0.5:1 molar ratio of anhydride to total amines present in the protein. The samples were vortexed and incubated on ice for 30 minutes. Following incubation, a 1 μL aliquot was removed from each reaction mix and was diluted individually in 57 μL of Buffer D (20 mM Tris-HCl, 0.1 mM EDTA, 100 mM KCl, 0.1% Triton X-100, 10% glycerol, pH 8.4), and saved on ice for later analysis (final concentration 0.67 μM). The above procedure was repeated 5 more times for each anhydride, so that the final products were 3 samples of P.a. RNase H2 that had reacted 6× with a 0.5 molar ratio of anhydride to primary amines, resulting in a cumulative treatment of a 3 molar ratio of anhydride to primary amines. After the 6^{th} cycle of anhydride treatment, 18.5 μL of 100 mM Tris-HCL (pH 8.4) was added to the three samples of modified RNase H2 protein to quench the reactions and prevent any further chemical modification from occurring (resulting in a final concentration of 20 mM Tris).

The bulk anhydride-treated sample was dialyzed into a buffer containing 20 mM Tris pH 8.4, 0.1 mM EDTA and 100 mM KCl using D-Tube™ Dialyzer Mini's (EMD Chemicals Inc., San Diego, Calif.) with a molecular weight cut-off of 6-8 kDa. Dialysis was performed at 4° C., 3×200 mL for 2 hours each, then 1×200 mL overnight, replacing with fresh buffer each time. After dialysis, protein concentration was verified by visualization on 4-20% SDS PAGE gels stained with Coomassie Brilliant Blue and comparison of the band intensity of the modified RNase H2 to BSA standards ranging from 100 to 600 ng. Gel images were quantified using ImageJ band-densitometry. The modified enzyme was stored at −20° C.

Analysis of modified RNase H2 enzyme activity. Enzymatic activity of unmodified and chemically-modified P.a. RNase H2 was measured using a synthetic fluorescence-quenched oligonucleotide reporter assay (FQ reporter assay). Sequence of the synthetic reporter is shown below.

SEQ ID NO. 3: RNH2 rC FAM-Reporter
FAM-CTCACTCAGAcCAGCATGATTTTTTCATGCTGGTCTGAGTGAG-FQ
SEQ ID NO. 4: RNH2 rC Competitor
CTCACTCAGAcCAGCATGATTTTTTCATGCTGGTCTGAGTGAG
DNA bases are uppercase, RNA bases are lowercase,
FAM is 6-carboxyfluorescein, and FQ is Iowa
Black® FQ dark quencher.

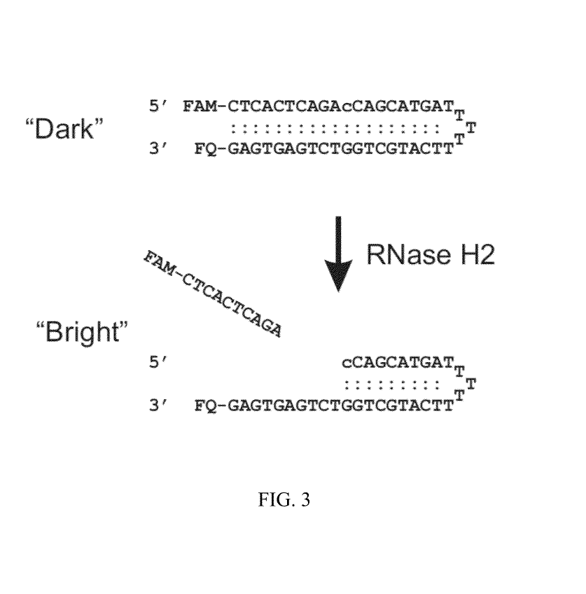

The reporter is a self-complementary sequence which forms a hairpin/loop structure with a 19-base stem domain and a 4 base loop domain. The molecule contains a FAM fluorescent dye at the 5′-end and a dark quencher at the 3′-end such that dye and quencher are brought into contact upon hairpin formation. In this configuration the fluorescent dye is quenched and the reporter is “dark”. A single ribonucleotide (rC) residue is positioned at position 11 from the 5′-end of the molecule, comprising an RNase H2 cleavage site. Following cleavage of the reporter molecule by RNase H2, the 10-base 5′-end fragment of the molecule dissociates, separating the fluorescent dye from the quencher. In this configuration, the dye is not quenched and the reporter is “bright”. A schematic representation of the RNase H2 activity assay is shown in FIG. 3.

The FQ reporter assay was used to compare activity of unmodified P.a. RNase H2 with aliquots previously “removed for later analysis” taken from the bulk enzyme modification reaction above. Reactivation of the anhydride-modified P.a. RNase H2 aliquots was similarly tested. Following each cycle of anhydride modification, 1 μL of each reaction was diluted with Buffer D, resulting in a final concentration of the enzyme of 667 nM (which would equal 200 mU/μL activity for the unmodified enzyme). These stocks were used either at this concentration or were further diluted to 9 nM concentration in Buffer D (which would equal 2.6 mU/μL activity for the unmodified enzyme). Aliquots of the unmodified enzyme and modified enzyme (at both 667 nM and 9 nM concentrations) were studied without additional treatment or were heated at 95° C. for 10 minutes prior to activity testing. Reactions were set up as follows: the FQ-reporter assays were done in 10 μL final volumes using 1 μL of the unmodified and modified enzyme dilutions; for the unmodified enzyme, the amount of enzyme employed corresponds to 200 mU or 2.6 mU of enzyme, respectively. Components of the FQ reporter assay are shown below in Table 1.

TABLE 1
Composition of the FQ reporter assay for RNase H2 activity

| Component | Final concentration | | |
|---|---|---|---|
| FAM-reporter oligo | 200 | nM | SEQ ID NO. 3 |
| Competitor oligo | 10 | μM | SEQ ID NO. 4 |
| KCl | 50 | mM | |
| Tris pH 8.4 | 20 | mM | |
| MgCl₂ | 1.5 | mM | |

| | |
|---|---|
| RNase H2 test sample | 67 nM or 0.9 nM (1 μL of the |
| | 667 or 9 nM dilutions) |
| Water | brine to final volume 10 μL |

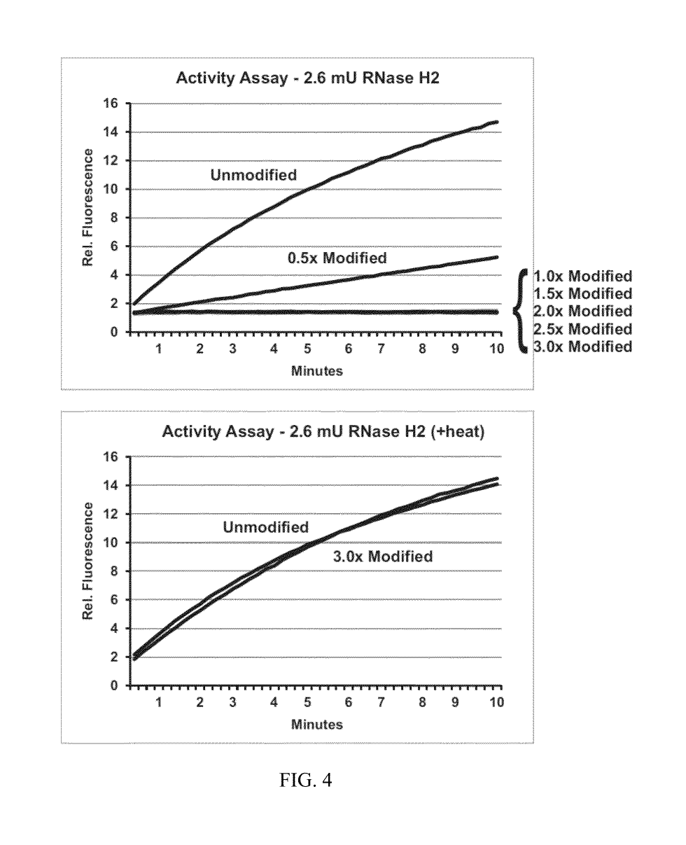

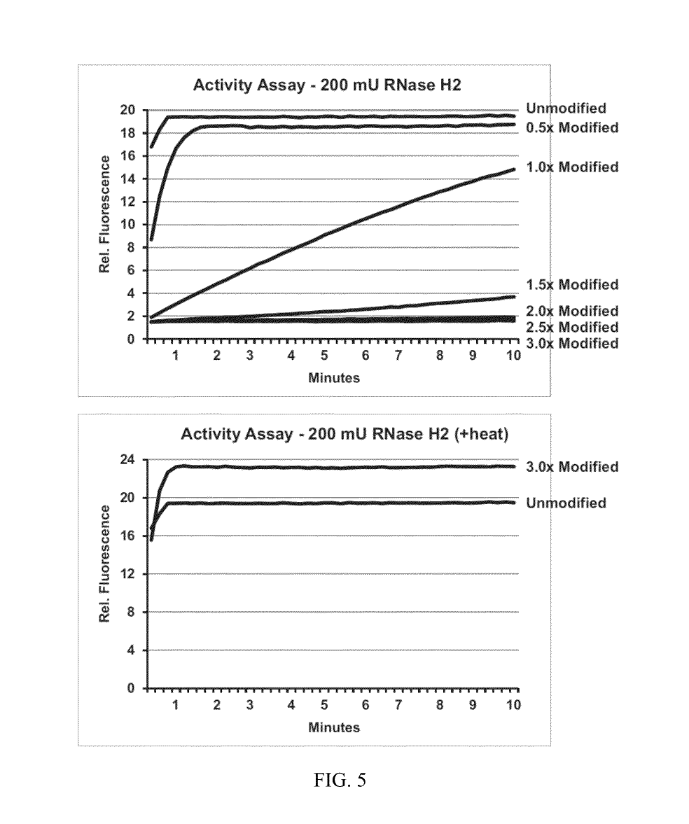

The 10 μL reactions were incubated in a 384-well plate in a Roche LightCycler® 480 (Roche Applied Science, Indianapolis, Ind., USA) at 60° C. for 10 minutes with a fluorescence measurement taken once every 11 seconds. Assays were run using unmodified P.a. RNase H2 and for all anhydride-treated samples (0.5×, 1.0×, 1.5×, 2.0×, 2.5×, and 3× modified) for each of the three anhydrides both before and after heat treatment at 95° C. for 10 minutes to reverse the modification. Results for the 2.6 mU assay of 3,4,5,6-tetrahydrophthalic anhydride-modified P.a. RNase H2 are shown in FIG. 4. A significant loss of activity was seen after the first treatment (0.5× modified) and no activity was detected for treatments 1.0×-3.0×. Complete return of enzymatic activity was seen after heat treatment for 10 minutes at 95° C., even for the most highly modified sample (3.0× modified). Results for the 200 mU assay of 3,4,5,6-tetrahydrophthalic anhydride-modified P.a. RNase H2 are shown in FIG. 5. Using this much higher concentration of enzyme, residual activity was seen in the 0.5×, 1.0×, and 1.5× treated samples however no activity was detected in the 2.0×, 2.5×, or 3.0× treated samples. Similar to the results obtained using 2.6 mU of enzyme, complete return of enzymatic activity was seen after heat treatment for 10 minutes at 95° C., even for the most highly modified sample (3.0× modified).

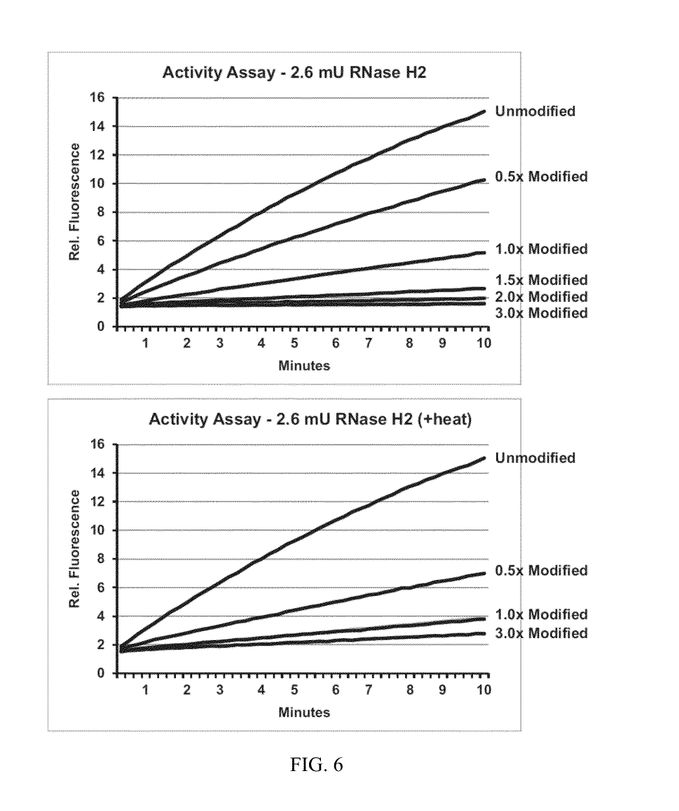

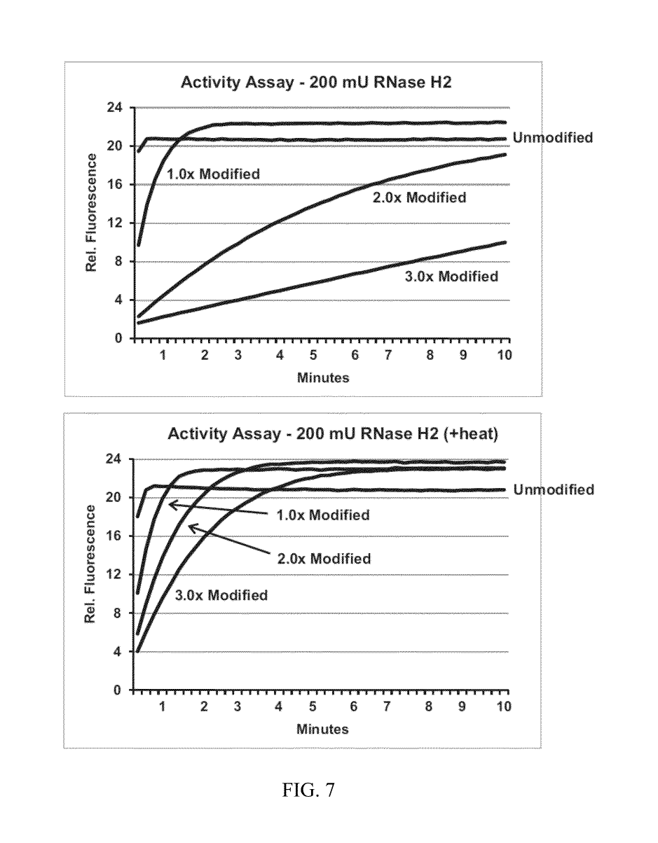

Results for the 2.6 mU assay of cis-aconitic anhydride-modified P.a. RNase H2 are shown in FIG. 6. Loss of activity was seen after the first treatment (0.5× modified) however complete inactivation of the enzyme was not achieved until the 2.0× level of modification. Unlike the results obtained using 3,4,5,6-tetrahydrophthalic anhydride, full activity did not return after heat treatment for 10 minutes at 95° C., even for the 0.5× treated sample. Extending the heat treatment to 15 minutes did not improve results. Results for the 200 mU assay of cis-aconitic anhydride-modified P.a. RNase H2 are shown in FIG. 7. Using this much higher concentration of enzyme, enzymatic activity was seen in all of the treated samples, indicating that complete inactivation of the enzyme was not achieved using this treatment protocol. As was seen for the 2.6 mU assays, none of the 200 mU assay samples returned to full activity following heat treatment.

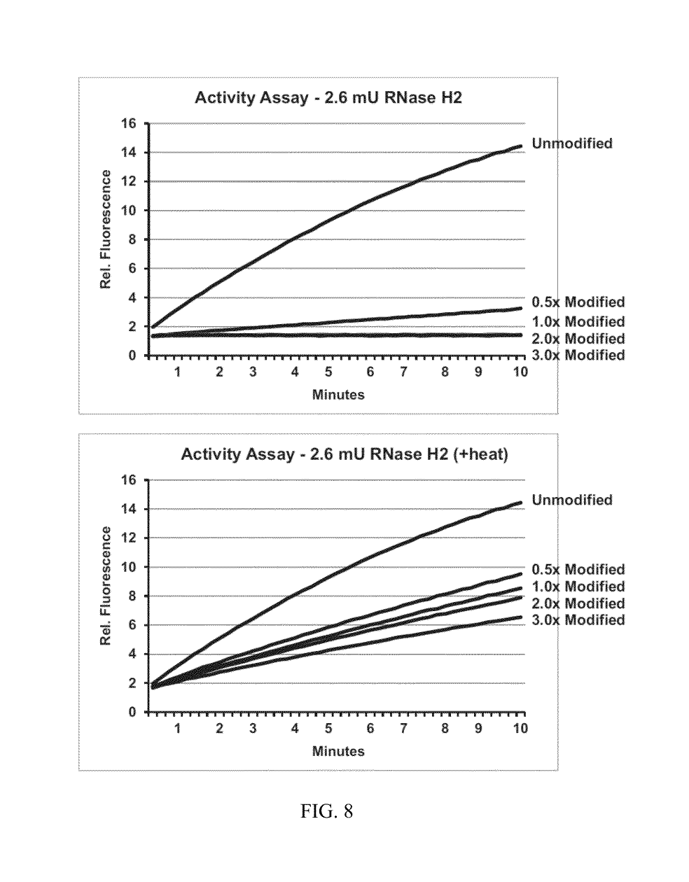

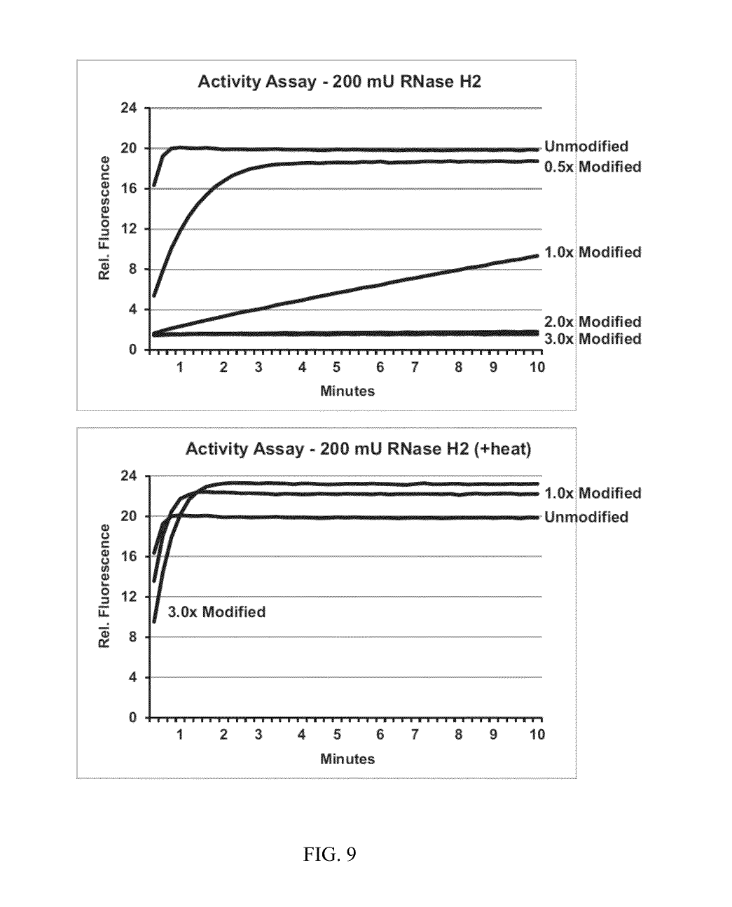

Results for the 2.6 mU assay of citraconic anhydride-modified P.a. RNase H2 are shown in FIG. 8. Loss of activity was seen after the first treatment (0.5× modified) and complete inactivation of the enzyme was achieved by the 1.0× level of modification. Like the results obtained using cis-aconitic anhydride, full activity did not return after heat treatment for 10 minutes at 95° C., even for the 0.5× treated sample. Extending the heat treatment to 15 minutes did not improve results. Results for the 200 mU assay of citraconic anhydride-modified P.a. RNase H2 are shown in FIG. 9. Using this much higher concentration of enzyme, enzymatic activity was detected in the 0.5×, 1.0×, and 1.5× treated samples, however complete inactivation was observed for the 2.0×-3.0× treated samples. For the 200 mU assay samples, enzyme activity almost completely returned after heat treatment, however a slightly slower rate of substrate cleavage was seen as the start of the reactions.

Treatment of P.a. RNase H2 using 3,4,5,6-tetrahydrophthalic anhydride, citraconic anhydride, or cis-aconitic anhydride will decrease enzyme activity and partial or full recovery of activity can be achieved with a short incubation at 95° C. This enzyme is extremely thermostable and can be incubated for periods of over 30 minutes without significant loss of activity, so anhydride-based inactivation/reactivation methods offer a suitable approach to make a hot-start RNase H2 enzyme. Of the various treatments tested, 3,4,5,6-tetrahydrophthalic anhydride showed the most favorable properties and treatment of the enzyme with a 2-fold molar excess of anhydride to free primary amines in the protein totally inactivated enzymatic activity. Further, the chemical modification was reversible with heat treatment at 95° C. for 10 minutes.

### Example 2

This example illustrates the spectrometry analysis of chemically-modified Pyrococcus abysii RNase H2.

The P.a. RNase H2 samples from Example 1 above were studied using electrospray ionization mass spectrometry (ESI-MS) to determine their molecular weights to determine the efficiency of chemical modification (inactivation) and the ability to remove the modifying groups by heat treatment (reactivation). Only the enzyme sample modified 6 times with a 0.5× molar ratio of 3,4,5,6-tetrahydrophthalic anhydride (final 3× molar ratio) was studied.

Mass spectrometry evaluation of modified P.a. RNase H2: Three samples of recombinant P.a. RNase H2 were prepared for mass spectrometry analysis in dialysis buffer, 20 mM Tris pH 8.4, 0.1 mM EDTA and 100 mM KCl:
1. Unmodified P.a. RNase H2 (control)
2. Modified (3× anhydride treated) P.a. RNase H2 (inactive)
3. Modified (3× anhydride treated) P.a. RNase H2, incubated at 95° C. for 10 minutes (active)

The three samples were examined at Novatia, LLC (Princeton, N.J., USA) with electrospray ionization mass spectrometry (ESI-MS). Reaction of each primary amine group in a protein with 3,4,5,6-tetrahydrophthalic anhydride will increase molecular weight by 152 Daltons, so reaction of all 29 amine groups in P.a. RNase H2 should increase mass by 4408 Daltons. The predicted molecular weights of the native and modified enzyme are shown in Table 2 below.

TABLE 2
Molecular weight predicted for recombinant P.a. RNase H2 before
and after reaction with 3,4,5,6-tetrahydrophthalic anhydride

| Sample | Expected mass | |
|---|---|---|
| Recombinant P.a. | 27,574 Daltons | unmodified control (active) |
| RNase H2 | | |
| Modified P.a. RNase H2 | 31,982 Daltons | modified (inactive) |
| Modified-heated P.a. | 27,574 Daltons | modified, reversed (active) |
| RNase H2 | | |

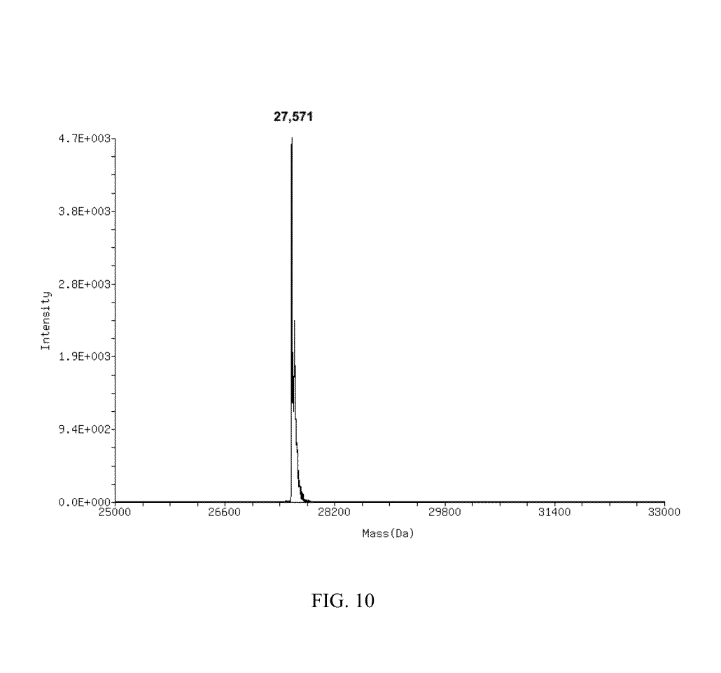

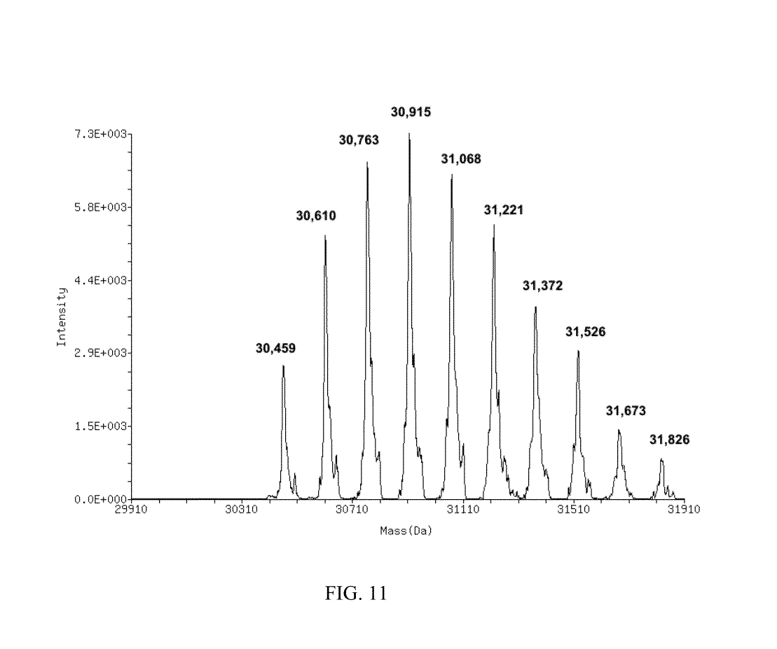

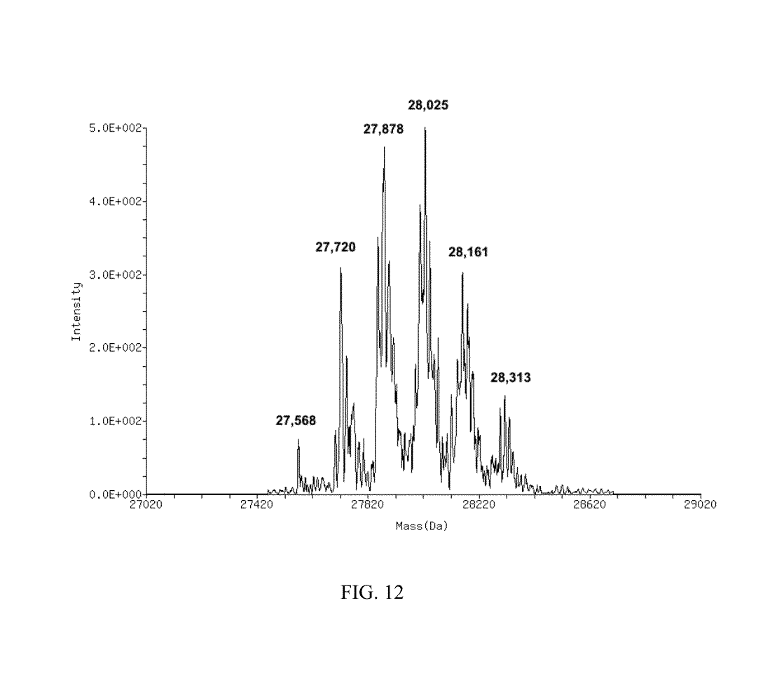

The deconvoluted ESI-MS spectra obtained for unmodified recombinant P.a. RNase H2 is shown in FIG. 10, for 3×-anhydride treated P.a. RNase H2 is shown in FIG. 11, and for heat-treated (reversed) 3×-anhydride treated P.a. RNase H2 is shown in FIG. 12. Mass values for the primary spectra peaks identified are summarized in Table 3 below. The unmodified enzyme showed a primary mass of 27,571 Da. The 3×-modified enzyme showed 10 mass peaks that correspond to protein species having 19 to 28 primary amines modified with 3,4,5,6-tetrahydrophthalic anhydride with the most prevalent species having 22 modified amines. The heat-treated 3×-modified enzyme showed 6 mass peaks that correspond to protein species having 0 to 5 primary amines modified with 3,4,5,6-tetrahydrophthalic anhydride with the most prevalent species having 3 modified amines.

TABLE 3
Summary of ESI-MS mass values obtained for P.a. RNase.

| | | No. of amines |
|---|---|---|
| Sample | Mass (Da) | modified |
| unmodified | 27,571 | 0 |
| 3x anhydride | 30,459 | 19 |
| treatment | 30,610 | 20 |
| | 30,763 | 21 |
| | 30,915 | 22 |
| | 31,068 | 23 |
| | 31,221 | 24 |
| | 31,372 | 25 |
| | 31,526 | 26 |
| | 31,673 | 27 |
| | 31,826 | 28 |
| 3x anhydride | 27,568 | 0 |
| treatment | 27,720 | 1 |
| followed by 10 | 27,878 | 2 |
| min. 95° C. heat | 28,025 | 3 |
| treatment | 28,161 | 4 |
| | 28,313 | 5 |

The heat-treated anhydride-modified P.a. RNase H2 showed around a 50% reduction in the rate of cleavage of the FQ-reporter oligonucleotide substrate compared with the unmodified enzyme, which correlated with retention of 0-5 modifying groups on amines on the mass spectra of this sample.

The unmodified RNase H2 sample displayed the expected mass. The RNase H2 sample modified to a final 3× molar ratio with 3,4,5,6-tetrahydrophthalic anhydride showed a 1000-fold reduction in activity which correlated with modification of a large fraction of the enzyme's primary amine groups. Heat treatment effectively reactivated the enzyme and near full activity was seen following 10 minutes incubation at 95° C. This treatment did not entirely remove all of the modified amine groups; however the reactivated enzyme functioned effectively in all biochemical performance tests performed.

### Example 3

The present example demonstrates that rhPCR using the anhydride-modified hot-start P.a. RNase H2 of the present invention performs well using native Taq DNA polymerase (non-hot-start DNA polymerase), even when reactions sit overnight at room temperature prior to commencing cycling, thus eliminating the need for use of a costly hot start DNA polymerase. The assays offer a comparison of PCR using unmodified vs. blocked-cleavable primers, native vs. hot-start Taq DNA polymerase, and native vs. hot-start P.a. RNase H2.

Methods. Quantitative real-time PCR (qPCR) was performed with 2 ng of human genomic DNA (GM18562, Coriell Institute for Medical Research, Camden, N.J., USA) using primers and a probe specific for a site in the human SMAD7 gene (rs4939827, NM_{—}005904). Reactions used either 0.4 U of a hot-start Taq DNA polymerase (iTaq™, Bio-Rad, Hercules, Calif., USA) or native Taq DNA polymerase (Enzymatics, Inc., Beverly, Mass., USA). Reactions contained iTaq™ buffer with 3 mM MgCl₂, 200 nM of each primer, 200 nM of a 5′-nuclease assay probe (SEQ ID NO. 9), 2 U of SUPERaseIn™ RNase inhibitor (Life Technologies, Carlsbad, Calif., USA), and 5 fmoles of P.a. RNase H2 (final concentration of 0.5 nM in a 10 μL reaction, or 2.6 mU of the unmodified enzyme). Either blocked-cleavable primers (SMAD7 For rC blocked, SEQ ID NO. 8 and SMAD7 Rev rG blocked, SEQ ID #6) or unmodified primers (SMAD7 For, SEQ ID NO. 7 and SMAD7 Rev, SEQ ID #5) were used. All oligonucleotides used in this study are shown below in Table 4. Reactions were either set up and run immediately or were set up and allowed to incubate at room temperature overnight before PCR cycling was started. Cycling was performed on a Roche LightCycler® 480 (Roche Applied Science, Indianapolis, Ind., USA) as follows: 95° C. for 10 minutes followed by 45 cycles of 95° C. for 10 seconds and 60° C. for 30 seconds. All reactions were performed in triplicate. The initial 10 minute incubation at 95° C. before thermocycling commences allows for reactivation of the hot-start DNA polymerase (iTaq) and the hot-start (anhydride-treated) P.a. RNase H2 enzymes. The native Taq DNA polymerase and the unmodified P.a. RNase H2 enzymes do not require this activation step, but all reactions were nevertheless run using the same cycling program.

TABLE 4
Synthetic oligonucleotide primers and probe employed in Example 3

| Name | Sequence | SEQ ID NO. |
|---|---|---|
| SMAD7 Rev | CTCACTCTAAACCCCAGCATT | SEQ ID NO. 5 |
| SMAD7 Rev rG | CTCACTCTAAACCCCAGCATTgGTCT-x | SEQ ID NO. 6 |
| blocked | | |
| SMAD7 For | CAGCCTCATCCAAAAGAGGAAA | SEQ ID NO. 7 |
| SMAD7 For rC | CAGCCTCATCCAAAAGAGGAAAcAGGA-x | SEQ ID NO. 8 |
| blocked | | |
| SMAD7 probe | FAM-CTCAGGAAACACAGACAATGCTGGG-IBFQ | SEQ ID NO. 9 |

DNA bases are uppercase and RNA bases are lowercase; x = C3 spacer (propanediol); FAM = 6-carboxyfluorescein; IBFQ = Iowa B1ack™ FQ fluorescence quencher

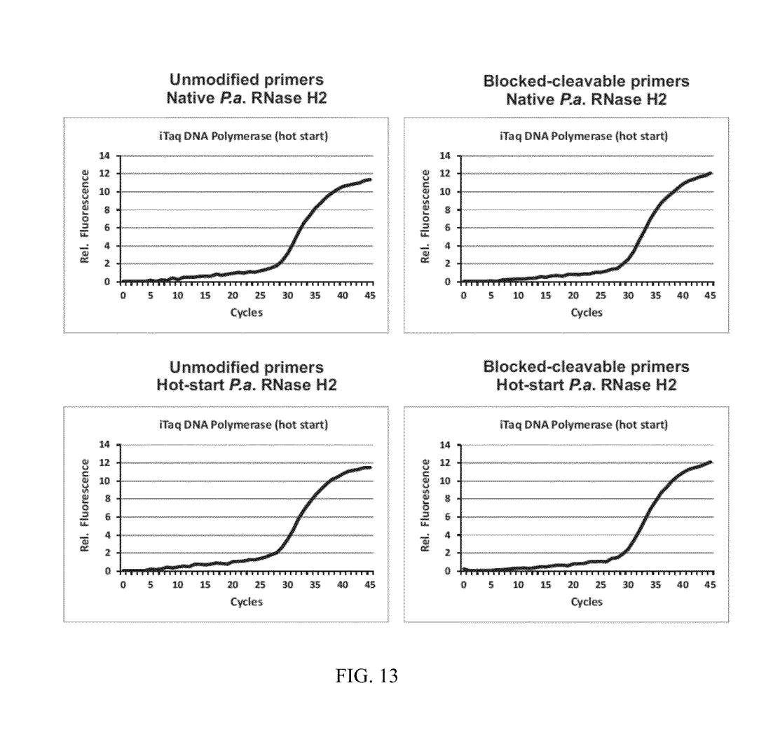

Results. When PCR was performed using a hot-start DNA polymerase, the amplification reactions proceeded with the same efficiency whether the reactions were run immediately following set up (not shown) or if the reactions were allowed to incubate overnight at room temperature prior to thermocycling. FIG. 13 shows amplification plots obtained using the hot start DNA polymerase iTaq following overnight incubation at room temperature. Use of unmodified primers (left panels) resulted in efficient amplification reactions and no difference was seen between addition of native P. a. RNase H2 (top left) and anhydride-modified hot start P.a. RNase H2 (bottom left). Use of blocked-cleavable primers (right panels) also resulted in efficient amplification reactions and no difference was seen between addition of native P.a. RNase H2 (top right) and anhydride-modified hot start P.a. RNase H2 (bottom right). No amplification occurred when using blocked-cleavable primers if RNase H2 was not added to the reactions (not shown).

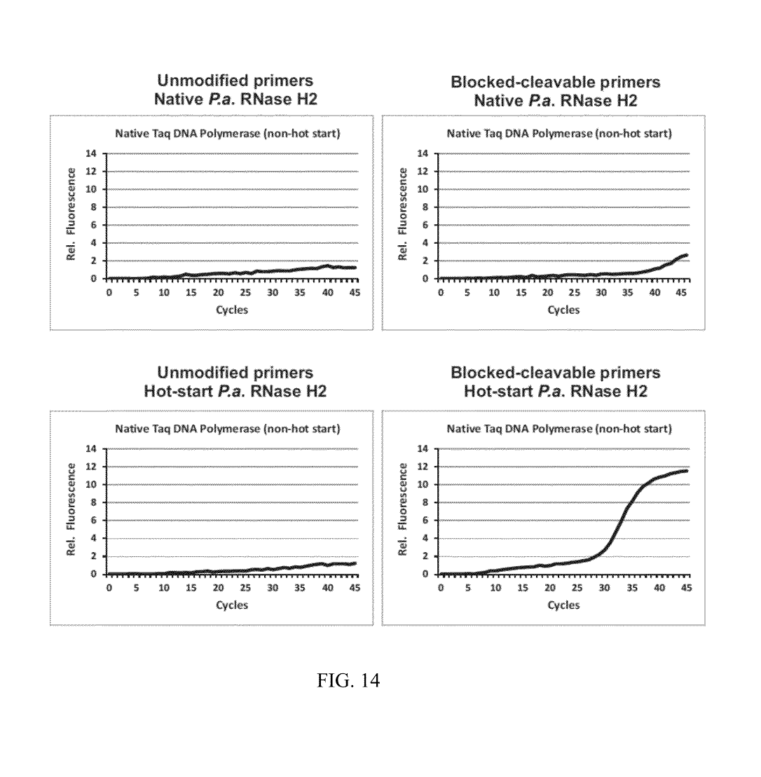

FIG. 14 shows amplification plots obtained using native Taq DNA polymerase. When the reactions were run immediately after all components were mixed, amplification occurred with the expected efficiency and the plots obtained were similar to those seen using a hot start DNA polymerase (not shown). In contrast, when the reaction plates were incubated overnight at room temperature prior to thermocycling, use of unmodified primers (left panels) resulted in no detectable amplification of the target nucleic acid sequence. In this case, active DNA polymerase was present with unblocked primers and undesired side reactions occurred at room temperature, consuming reagents and compromising the quality of the subsequent desired amplification reaction. Use of blocked-cleavable primers (right panels) resulted in efficient amplification of the target nucleic acid when anhydride-modified hot start P.a. RNase H2 was employed for primer activation (bottom right) but not when native P.a. RNase H2 was employed (top right). Thus, even though P.a. RNase H2 has very little activity at room temperature, sufficient activity remains that a modified hot start version of the enzyme is needed when performing rhPCR under these conditions.

Undesired reactions occur in amplification reactions at room temperature that are dependent upon the presence of an active DNA polymerase and primers in the reaction. These reactions consume reaction components and reduce the efficiency and quality of the desired amplification reaction. Use of a costly hot-start DNA polymerase can eliminate these artifacts. Alternatively, use of rhPCR with blocked-cleavable primers and an anhydride-modified hot-start RNase H2 enzyme can be used and yield efficient, specific amplification reactions.

### Example 4

The following example illustrates the use of 3,4,5,6-tetrahydrophthalic anhydride-modified hot-start P.a. RNase H2 in single-tube high-temperature RT-qPCR reactions.

P.a. RNase H2 has minimal activity at low temperatures (e.g., 25-45° C.) and the reduction in enzyme activity in the conditions used in low temperature RT reactions may be sufficient to allow this enzyme to be present during RT. A 2-step low temperature RT reaction was done with and without P.a. RNase H2 present using an internal gene-specific primer, random hexamer primers, or oligo-dT primers. Following cDNA synthesis, qPCR was performed to amplify a 157 bp region within the human tumor necrosis factor receptor superfamily member 1A (TNFRSF1A, NM_{—}001065). Sequences of the primers, probe, and target nucleic acid employed are shown below in Table 5. Note that the PCR assay is located 1509 bases 5′- to the poly-A tail site of this gene.

TABLE 5
Sequences of primers employed in TNFRSF1A RT-qPCR experiments

| Name | Sequence | SEQ ID NO. |
|---|---|---|
| TNFRSF1A | AAACCTTTTCCAGTGCTTCAATTGCAGCCTCTGCCTCAATGGGACC | SEQ ID NO. 10 |
| target | GTGCACCTCTCCTGCCAGGAGAAACAGAACACCGTGTGCACCTGCC | |
| | ATGCAGGTTTCTTTCTAAGAGAAAACGAGTGTGTCTCCTGTAGTAA | |
| | CTGTAAGAAAAGCCTGGAGTGCACGAAGTTGTGCCTACCCCAGATT | |
| | GAGAATGTTAAGGGCACTGAGGACTCAGGCACCACAGTGCTGTTGC | |
| | CCCTGGTCATTTTCTTTGGTCTTTGCCTTTTATCCCTCCTCTTCAT | |
| | TGGTTTAATGTATCGCTACCAACGGTGGAAGTCCAAGCTCTACTCC | |
| | ATTGTTTGTGGGAAATCGACACCTGAAAAAGAGGGGGAGCTTGAAG | |
| | GAACTACTAC | |
| TNFRSF1A | AAACCTTTTCCAGTGCTTCA | SEQ ID NO. 11 |
| 695 For | | |
| TNFRSF1A | CTCCAGGCTTTTCTTACAGT | SEQ ID NO. 12 |
| 832 Rev | | |
| TNFRSF1A | FAM-CCGTGCACCTCTCCTGCCAG-IBFQ | SEQ ID NO. 13 |
| 739 Probe | | |
| TNFRSF1A | GTAGTAGTTCCTTCAAGCTC | SEQ ID NO. 14 |
| 1079 RT | | |
| Random | NNNNNN | SEQ ID NO. 15 |
| Hex | | |
| Oligo-dT | TTTTTTTTTTTTTTTTTT | SEQ ID NO. 16 |

Sites of the For (forward) and Rev (reverse) priming sites are underlined in the TNFRSF1A target nucleic acid sequence.
The primer binding site for the gene-specific RT primer is shown in italics and is also underlined.
FAM = 6-carboxyfluorescein and IBFQ = Iowa Black®-FQ fluorescence quencher.

Reverse transcription was performed using 150 ng HeLa cell total RNA in a 15 μL reaction with 1× first-strand buffer (50 mM Tris-HCl, pH 8.3 at room temperature; 75 mM KCl; 3 mM MgCl₂), 0.01 mM DTT, 1 mM dNTPs, 30 U Superscript-II RT, 5 U SUPERase-In™ RNase inhibitor and either 1.3 μM of the TNFRSF1A-specific RT primer (SEQ ID NO. 14), 250 ng oligo-dT primer (SEQ ID NO. 16), or 250 ng random hexamer primer (SEQ ID NO. 15). Reactions were run either with or without the addition of 2.6 mU of unmodified recombinant P.a. RNase H2 at 42° C. for 60 minutes, followed by a 15 minute RT enzyme inactivation step at 70° C.

Amplification reactions were run using 2 μL of each of the above RT reactions (e.g., cDNA made from 20 ng of total cellular RNA). Reactions comprised 1× Immolase reaction buffer (16 mM (NH₄)₂SO₄, 100 mM Tris-HCL pH 8.3, and 0.01% Tween-20), 0.4 U 1 mmolase DNA polymerase (Bioline, Taunton, Mass., USA), 3 mM MgCl₂, 800 μM dNTPs, 200 nM forward and reverse primers (SEQ ID NOs. 11 & 12), and 200 nM probe (SEQ IN NO. 13) in a final 10 μL reaction volume. PCR cycling conditions employed were: 95° C. for 5 minutes followed by 45 cycles of 2-step PCR with 95° C. for 15 seconds and 60° C. for 60 seconds. Reactions were run on a Roche LightCycler® 480 (Roche Applied Science, Indianapolis, Ind., USA) thermocycler. All reactions were run in triplicate. The quantification cycle value (Cq) was determined using the absolute quantification/2^{nd} derivative method.

Results of PCR amplification of the TNFRSF1A gene from cDNA made using low temperature RT with and without P.a. RNase H2 are shown in Table 6 below. In the absence of RNase H2, all three RT-primer variations yielded similar results, having Cq values in the 25-26 cycle range. In the presence of RNase H2, the target levels detected in the RT reactions primed using the gene specific primer or random hexamers were nearly identical to the “minus RNase H2” control reactions; however, the RT reaction primed using oligo-dT showed a 2 cycle delay, indicating slightly lower levels of target were present in this RT reaction. Thus the presence of P.a. RNase H2 in an RT reaction performed at 42° C. did not adversely affect the level of target cDNA made when the RT primers were located near the PCR assay site (gene specific primer and random hexamers) but did result in a less efficient RT reaction when the RT primer was located 1509 bases from the site of the PCR assay (oligo-dT). Presumably this is due to partial degradation of the RNA in the RNA:DNA heteroduplex present during cDNA synthesis which only impacted the sensitivity of the reaction when long cDNA extension was required. Degradation of the RNA template would increase if the reaction was performed at a higher temperature where the P.a. RNase H2 has higher activity (e.g., 55-65° C.).

TABLE 6
Amplification of a cDNA target made using low temperature
RT with or without P.a. RNase H2 present.

| RT primer employed | | −RNase H2 | +RNase H2 |
|---|---|---|---|
| TNFRSF1A 1079 RT | SEQ ID NO. 14 | 26.1 | 26.7 |
| Random Hexamer | SEQ ID NO. 15 | 25.0 | 25.7 |
| Oligo-dT | SEQ ID NO. 16 | 25.9 | 27.7 |

The cycle quantification value (Cq) where fluorescence signal from the qPCR first is detectable is shown.

RT can be performed at elevated temperatures using a thermostable reverse transcriptase. High temperature RT methods allow for higher fidelity cDNA synthesis from RNA templates that have complex, stable secondary structures that interfere with the processivity of the DNA polymerase at lower temperatures. One example of this approach employs the HawkZ05™ RT enzyme (Roche Applied Science, Indianapolis, Ind., USA). When using manganese as the divalent cation instead of magnesium, this enzyme functions as both a thermostable reverse transcriptase and a DNA polymerase which can support both steps of RT-qPCR. Note that P.a. RNase H2 functions well in the presence of either Mn++ or Mg++ cations and will have good catalytic activity in the reaction conditions employed in this example. Reactions are typically done in a closed-tube format where both the RT and PCR steps are sequentially performed in a single tube. This approach limits the aerosol spread of reaction products that inevitably occurs when reaction tubes are opened to transfer products, thereby reducing the risk of cross-contamination and false-positive reactions, a particularly important feature for molecular diagnostic applications.

Amplification efficiency at a site in the human SFRS9 gene (NM_{—}003769) was studied using high temperature RT-qPCR without addition of RNase H2, with the addition of native P.a. RNase H2, or with the addition of 3,4,5,6-tetrahydrophthalic anhydride—modified P.a. RNase H2 (see Example 1 above). The anhydride-modified P.a. RNase H2 will also be referred to as the “hot-start RNase H2” or the “HS-RNase H2”. Standard methods for single-tube high temperature RT-qPCR employ unmodified primers with a fluorescence-quenched 5′-nuclease reporter probe; the RT reaction is primed by the reverse PCR primer. The present experiment was done using either unmodified primers (SFRS9 For and Rev, SEQ ID NOs. 17 &18) or with a blocked-cleavable forward primer (SFRS9 For blocked, SEQ ID NO. 19) paired with the unmodified Rev primer (SEQ ID NO. 18). Note that the blocked-cleavable For primer requires activation by RNase H2 to function in PCR. Use of the blocked-cleavable forward primer in place of an unmodified For primer will increase reaction specificity and could be used to selectively amplify one allele if SNP discrimination was desired (see Example 5). Note that the forward primer has no function during the RT phase of the reaction and so does not need to be cleaved (activated) by RNase H2 until the PCR phase of the reaction begins. Oligonucleotide sequences are shown in Table 7 below.

TABLE 7
Sequences of primers employed in the SFRS9 RT-qPCR experiments

| Name | Sequence | SEQ ID NO. |
|---|---|---|
| SFRS9 For | TGTGCAGAAGGATGGAGT | SEQ ID NO. 17 |
| SFRS9 Rev | CTGGTGCTTCTCTCAGGATA | SEQ ID NO. 18 |
| SFRS9 For blocked | TGTGCAGAAGGATGGAGTgGxxA | SEQ ID NO. 19 |
| SFRS9 probe | FAM-TGGAATATGCCCTGCGTAAACTGGA- | SEQ ID NO. 20 |
| | IBFQ | |

DNA bases are uppercase; RNA bases are lowercase; FAM = 6-carboxyfluorescein; IBFQ = Iowa Black®-FQ fluorescence quencher; x = C3 spacer (propanediol).

RT-qPCR was performed using 20 ng of HeLa cell RNA per 10 μL reaction with the HawkZ05™ Fast One-Step RT-PCR Master Mix (Roche Applied Science, Indianapolis, Ind., USA), 1.5 mM Mn(OAc)₂, 200 nM primers, and 200 nM probe. 2.6, 25, or 200 mU of unmodified P.a. RNase H2 or the new HS-P.a. RNase H2 was added to each reaction; control reactions without RNase H2 were also performed. Reactions used either unmodified primers (SFRS9 For and SFRS9 Rev, SEQ ID NOs. 17 &18) or the blocked forward primer and unmodified reverse primer (SFRS9 For blocked and SFRS9 Rev, SEQ ID NOs. 19 &18) with the SFRS9 probe (SEQ ID NO. 20) in a 5′-nuclease assay format.

The RT phase of the reaction proceeded during the first 15 minutes of incubation which was done stepwise at 55° C. for 5 minutes, 60° C. for 5 minutes, and 65° C. for 5 minutes. The target nucleic acids were then denatured with incubation at 95° C. for 10 minutes after which PCR was run for 45 cycles of 92° C. for 5 seconds, 60° C. for 40 seconds, and 72° C. for 1 second. Reactions were run on a Roche LightCycler® 480 (Roche Applied Science, Indianapolis, Ind., USA) thermocycler. All reactions were performed in triplicate. Note that the 95° C. incubation also activates the HS-P.a. RNase H2 enzyme.

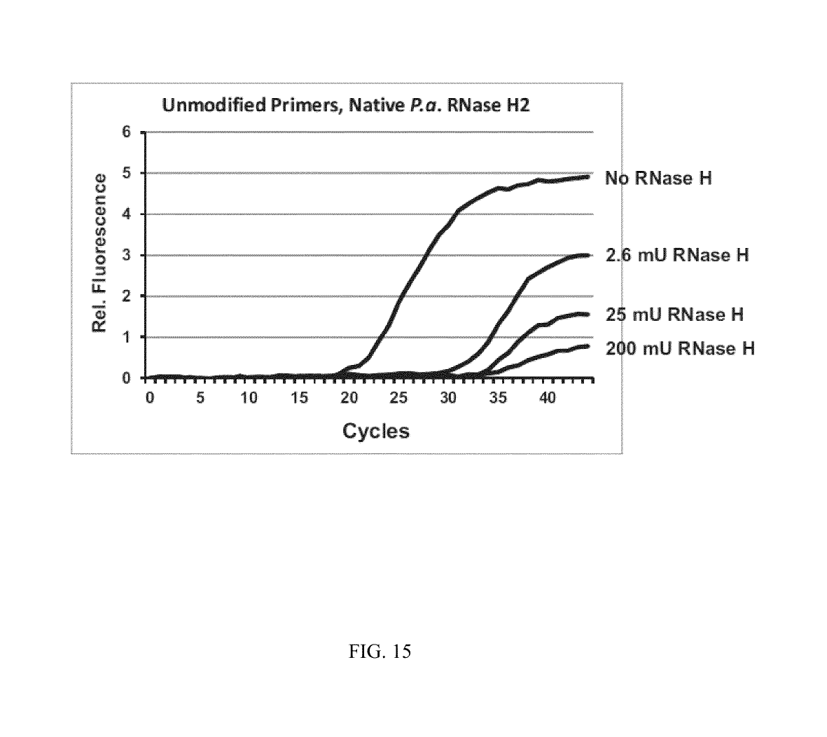

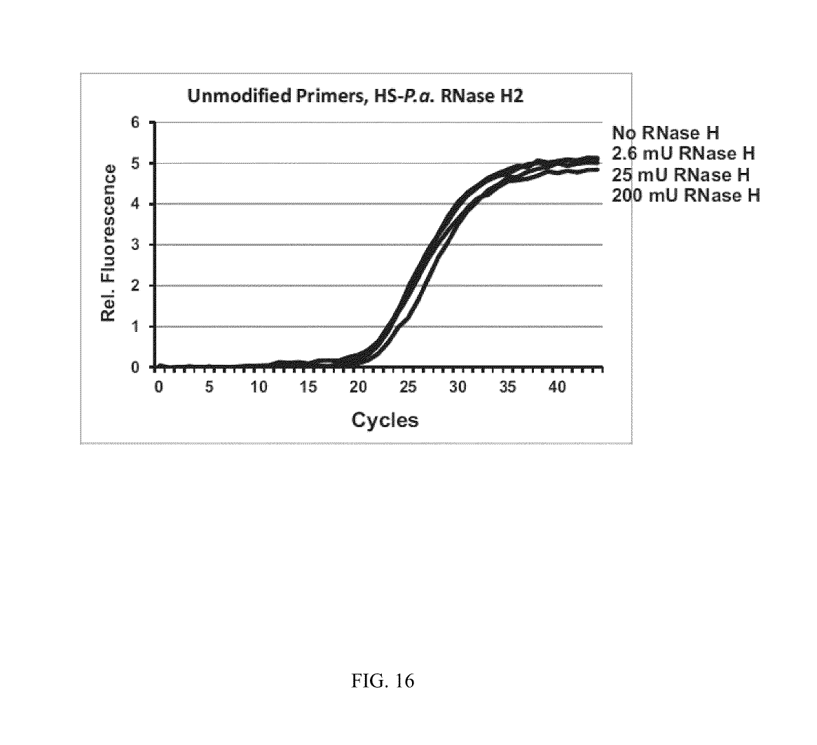

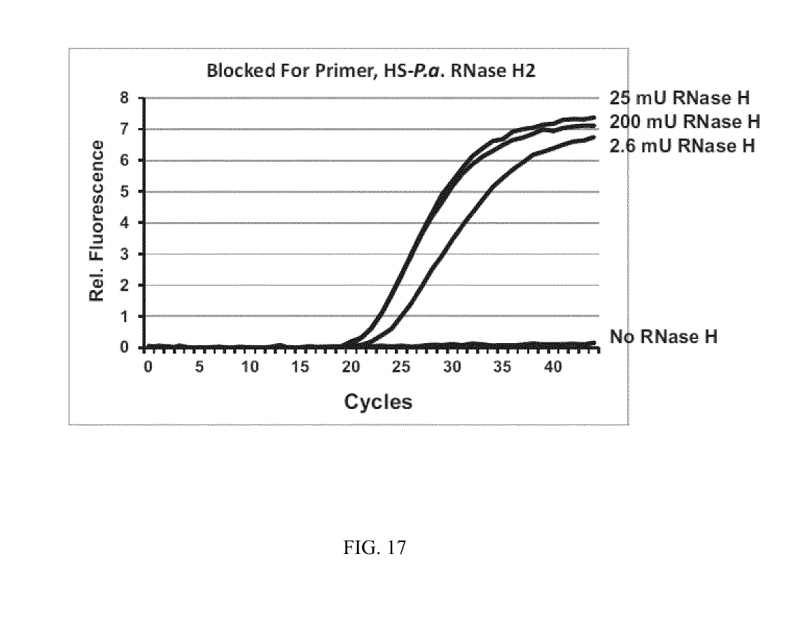

Results are shown in FIGS. 15-17. For the reactions done using unmodified primers, the single-tube high temperature RT-qPCR reaction performed well without RNase H2 present. Addition of even small amounts of native P.a. RNase H2, however, had a deleterious impact on the reaction (FIG. 15). Addition of 2.6 mU of enzyme shifted the Cq value by ˜10 cycles, addition of 25 mU of enzyme shifted the Cq value by ˜14 cycles, and reactions done with 200 mU of enzyme showed no appreciable amplification. At the reaction temperatures used for the RT reaction (55°-65° C.), P.a. is highly active and most likely degraded the RNA template during the early phase of cDNA synthesis. In contrast, reactions performed using the anhydride-modified HS-P.a. RNase H2 showed amplification of the SFRS9 target with similar efficiency to reactions done in the absence of RNase H2 (FIG. 16). In this case, no differences were seen between reactions done without RNase H2 or with 2.6 to 200 mU of RNase H2, confirming that the modified HS-P.a. RNase H2 enzyme was sufficiently inactivated to not degrade the RNA target during cDNA synthesis, even when performed at 55°-65° C. Results for reactions performed using the blocked-cleavable For PCR primer with an unmodified Rev PCR primer are shown in FIG. 17. Consistent with the need for cleavage/activation of the blocked primer, no amplification was seen in the absence of RNase H2. Using the 3,4,5,6-tetrahydrophthalic anhydride—modified HS-P.a. RNase H2, reactions with 25 mU or 200 mU of enzyme showed amplification efficiencies identical to that seen using unmodified primers. Reactions done using 2.6 mU of enzyme showed around a 4 cycle delay, indicating that the reaction conditions did not produce complete primer cleavage.

The presence of native P.a. RNase H2 in high-temperature RT-qPCR reactions degrades the RNA during cDNA synthesis, preventing amplification. Use of the new anhydride-modified P.a. RNase H2 of the present invention allows for the enzyme to be present during the RT reaction in an inactive state, so cDNA synthesis proceeds normally. The heat denaturation step done after cDNA synthesis activates the modified RNase H2, after which rhPCR can be performed using blocked-cleavable primers. Therefore the higher specificity of rhPCR can be adapted to high-temperature, single-tube RT-qPCR.

### Example 5

The following example illustrates the utility of 3,4,5,6-tetrahydrophthalic anhydride—modified P.a. RNase H2 in a RT-qPCR single-nucleotide polymorphism (SNP) assay. The assays of this example will demonstrate the discrimination of KRAS SNPs by single-tube RT-rhPCR.

A G/T SNP site in the human KRAS gene (NM_{—}004985) was studied using rhPCR and the high-temperature single-tube RT-qPCR HawkZ05™ Fast One-Step RT-PCR Master Mix (Roche Applied Science, Indianapolis, Ind., USA) using methods similar to those described in Example 4 above.

RT-qPCR was performed using 50 ng HCT-15 (G/G) or SW480 (T/T) total cellular RNA per 10 μL reaction with HawkZ05™ Fast One-Step RT-PCR Master Mix, 1 mM Mn(OAc)₂, 200 nM primers, and 200 nM probe. 200 mU of HS-P.a. RNase H2 was added to each reaction before RT and PCR were performed. All reactions employed the same unmodified KRAS Rev primer (SEQ ID NO. 21), which served as both the RT primer and the reverse PCR primer. Some reactions paired the KRAS Rev primer with an unmodified non-discriminatory KRAS For primer (SEQ ID NO. 22). Other reactions paired the KRAS Rev primer with either a G-SNP discriminatory KRAS rG For blocked-cleavable primer (SEQ ID NO. 23) or a T-SNP discriminatory KRAS rU For blocked-cleavable primer (SEQ ID NO. 24). All assays employed the same fluorescence-quenched probe (SEQ ID NO. 25) as a 5′-nuclease assay reporter. Primers and probes employed in Example 5 are shown in Table 8 below.

TABLE 8
KRAS-specific primers and probes

| Name | Sequence | SEQ ID NO. |
|---|---|---|
| KRAS Rev | TCTATTGTTGGATCATATTCGTCCACA | SEQ ID |
| | | NO. 21 |
| KRAS For | AACTTGTGGTAGTTGGAGCTG | SEQ ID |
| | | NO. 22 |
| KRAS rG | AACTTGTGGTAGTTGGAGCTGgTxxC | SEQ ID |
| For | | NO. 23 |
| KRAS rU | AACTTGTGGTAGTTGGAGCTGuTxxC | SEQ ID |
| For | | NO. 24 |
| KRAS | FAM-AGAGTGCCTTGACGATACAGC-IBFQ | SEQ ID |
| Probe | | NO. 25 |

DNA bases are uppercase; RNA bases are lowercase; FAM = 6-carboxyfluorescein; IBFQ = Iowa Black®-FQ fluorescence quencher; x = C3 spacer (propanediol).

The RT phase of the reaction proceeded during the first 15 minutes of incubation which was done stepwise at 55° C. for 5 minutes, 60° C. for 5 minutes, and 65° C. for 5 minutes. The target nucleic acids were then denatured with incubation at 95° C. for 10 minutes after which PCR was run for 45 cycles of 92° C. for 5 seconds, 60° C. for 40 seconds, and 72° C. for 1 second. Reactions were run on a Roche LightCycler® 480 (Roche Applied Science, Indianapolis, Ind., USA) thermocycler. All reactions were performed in triplicate. Note that the 95° C. incubation also activates the HS-P.a. RNase H2 enzyme.

Results are shown in Table 9 below. Reactions performed using the unmodified non-discriminatory KRAS For primer showed similar Cq values for both cell lines. The blocked-cleavable KRAS rG primer showed a Cq of 25.9 using HCT-15 DNA (G/G) but was delayed 12.3 cycles to 38.2 using SW480 DNA (T/T). Conversely, the blocked-cleavable KRAS rU primer showed a delayed Cq of 36.8 using HCT-15 DNA (G/G) and a Cq of 25.9 using SW480 DNA (T/T).

TABLE 9
Mismatch discrimination in the KRAS RT-qPCR SNP assay

| | Cq Values | | |
|---|---|---|---|
| For Primer | HCT-15 (G/G) | SW480 (T/T) | ΔCq |
| KRAS For | 24.8 | 24.1 | — |
| KRAS rG | 25.9 | 38.2 | 12.3 |
| KRAS rU | 36.8 | 25.9 | 10.8 |

Use of the anhydride-modified HS-P.a. RNase H2 enables a highly accurate SNP rhPCR assay to be performed in a single-tube high-temperature RT-qPCR format, demonstrating utility of the modified enzyme used in the methods of the present invention.

### Example 6

This example demonstrates a method to utilize two blocked PCR primers with an external RT primer in one-tube RT-qPCR. To eliminate the possibility that amplification products will originate from the RT primer, the RT primer is modified such that it retains the capacity to prime cDNA synthesis but does not support PCR.

Examples 4 and 5 demonstrate that anhydride-modified HS-P.a. RNase H2 can be present in high temperature RT reactions and that the inactivated enzyme does not degrade the RNA template during cDNA synthesis. The examples further demonstrate that the enzyme is reactivated with incubation at 95° C. for 10 minutes, after which rhPCR can be performed using blocked-cleavable primers. In these examples, the reverse PCR primer was unmodified and also functioned as a gene-specific RT primer. If additional specificity is desired through use of a blocked-cleavable reverse primer (in place of the unmodified reverse primer), it becomes necessary to add a third primer oligonucleotide to the reaction to function as the RT primer, since the PCR reverse primer is now blocked. The new RT primer is placed 3′- to the PCR reverse primer. However, being unmodified, this primer can participate in the PCR reaction, eliminating any specificity improvements gained from use of the blocked-cleavable reverse primer. It is therefore desirable to modify the RT primer so that it can prime cDNA synthesis in the RT reaction but does not participate in subsequent amplification reactions. One approach is to make an RT primer having a lower melting temperature (Tm) than the PCR primers so that the RT reaction could, for example, proceed at 60° C. while the amplification reaction proceeds at 70° C., i.e., PCR is run at a temperature sufficiently above the Tm of the RT primer that this primer no longer anneals to template. However, the high temperature RT protocol in use in commercial high-temperature RT methods typically involves incubation up to 65° C. to disrupt RNA secondary structure, and most PCR reactions are designed with primer annealing to occur at or around 60° C. Thus while use of differential Tm for RT vs. PCR primers could be employed, this method requires redesign of PCR primers and reactions to operate at higher temperatures. The present example demonstrates methods to use modified RT primers in standard reaction temperature wherein the RT primer is competent to primer cDNA synthesis but does not participate in the subsequent amplification reaction.

Two modification strategies are described which achieve the same goal. First, a cleavable linkage is included internally within the RT primer. The RT reaction (cDNA synthesis) is performed with the primer intact, after which a chemical or enzymatic event cleaves the RT primer at the scissile linkage. The remaining primer fragments no longer have sufficient binding affinity to primer further DNA synthesis reactions at the reaction temperatures commonly used in PCR. A variety of approaches can be used to introduce a cleavage site in the primer, which are well known to those with skill in the art, such as linkages susceptible to chemical cleavage, restriction enzyme sites, and the like. In the present example, a single RNA base is placed at or around the middle of the RT primer. When using anhydride-modified HS-P.a. RNase H2, the RNase H2 is inactive during cDNA synthesis and the primer functions normally. After cDNA synthesis, the reaction is heated at 95° C. for around 10 minutes and the HS-P.a. RNase H2 is reactivated. When the reaction returns to 50-70° C. during PCR, the RT primer itself becomes a substrate for RNase H2 attach. The RT primer is cleaved, and the resulting short fragments now have a lowered Tm and cannot participate in amplification reactions in the 50-70° C. range. Thus the RT primer serves to prime cDNA synthesis but does not participate in PCR.

Second, modifying group is placed at or around the center of the RT primer which does not affect the ability of the oligonucleotide to prime DNA synthesis but which impairs its ability to function as a template for DNA synthesis. Thus linear primer extension reactions are supported (such as cDNA synthesis), but exponential amplification reactions are prevented; during subsequent cycles of amplification, the extension product prematurely terminates at the site of the primer blocking group with the result that the final amplification product is shortened and does not contain a primer binding site of sufficient length for the RT primer to bind at reaction temperatures in the 50-70° C. range. A variety of blocking groups that can serve this purpose are known to those with skill in the art, such as 2′-modified RNA residues (e.g., 2′-O-methyl RNA), abasic residues (aliphatic spaces, d-spacer), unnatural bases (e.g., 5-nitroindole), and the like (see Behlke et al., U.S. Pat. Nos. 7,112,406 and 7,629,152). The present example employs a non-nucleotide napthyl-azo modifier as the blocking group (see Laikhter et al., U.S. Pat. No. 8,084,588 and Rose et al., U.S. Patent Application 2011/0236898), which has the advantage of blocking template function (i.e., inducing chain termination) while not destabilizing hybridization of the modified primer to the target nucleic acid. Many of the modifying groups which disrupt template function, such as aliphatic spacers, d-spacers, and the like, also impair duplex formation (e.g., lower Tm of the primer).

Methods. RT-qPCR was performed using 10 ng HeLa cell total RNA per 10 μL reaction with HawkZ05™ Fast One-Step RT-PCR Master Mix (Roche Applied Science, Indianapolis, Ind., USA), 1.5 mM Mn(OAc)₂, 200 nM PCR primers, and 200 nM probe (SFRS9 probe, SEQ ID NO. 20). External RT primers were used at 200 nM, 100 nM, 50 nM, 10 nM, or 0 nM. Either no RNase H2 or 10 mU of 3,4,5,6-tetrahydrophthalic anhydride-modified HS-P.a. RNase H2 was added to each reaction. Amplification was performed using either unmodified PCR primers (SFRS9 For and Rev, SEQ ID NOs. 17 and 18) or blocked-cleavable rhPCR primers (SFRS9 For rG and SFRS9 Rev rA, SEQ ID NOs. 27 and 28). The RT phase of the reaction was performed using either no external RT primer, an unmodified primer (SFRS9-RT, SEQ ID NO. 31), a modified primer having an internal RNA residue (SFRS9-RT-rC, SEQ ID NO. 30), or a modified primer having an internal non-nucleotide napthyl-azo modifier (SFRS9-RT-ZEN, SEQ ID NO. 29). Sequences are shown in Table 10 below.

TABLE 10
Sequences of SFRS9 primers employed in Example 6

| Name | Sequence | SEQ ID NO. |
|---|---|---|
| SFRS9 target | TGTGCAGAAGGATGGAGTGGGGATGGTCGAGTATCTCAGAA | SEQ ID NO. 26 |
| | AAGAAGACATGGAATATGCCCTGCGTAAACTGGATGACACC | |
| | AAATTCCGCTCTCATGAGGGTGAAACTTCCTACATCCGAGT | |
| | TTATCCTGAGAGAAGCACCAGCTATGGCTACTCACGGTCTC | |
| | GGTCT | |
| SFRS9 For | TGTGCAGAAGGATGGAGT | SEQ ID NO. 17 |
| SFRS9 Rev | CTGGTGCTTCTCTCAGGATA | SEQ ID NO. 18 |
| SFRS9 For rG | TGTGCAGAAGGATGGAGTgGGGA-x | SEQ ID NO. 27 |
| SFRS9 Rev rA | CTGGTGCTTCTCTCAGGATAaACTC-x | SEQ ID NO. 28 |
| SFRS9 probe | FAM-TGGAATATGCCCTGCGTAAACTGGA-IBFQ | SEQ ID NO. 20 |
| SFRS9-RT-ZEN | AGACCGAGAC(Z)GTGAGTAGCC | SEQ ID NO. 29 |
| SFRS9-RT-rC | AGACCGAGACcGTGAGTAGCC | SEQ ID NO. 30 |
| SFRS9-RT | AGACCGAGACCGTGAGTAGCC | SEQ ID NO. 31 |

Sites of the For (forward) and Rev (reverse) priming sites are underlined in the SFRS9 target nucleic acid sequence.
The primer binding site for the gene-specific RT primer is shown in italics and is also underlined.
DNA bases are uppercase and RNA bases are lowercase.
FAM = 6-carboxyfluorescein, IBFQ = Iowa Black®-FQ fluorescence quencher, x = C3 spacer (propanediol), and (z) = internal napthyl-azo modifier.

The RT phase of the reaction proceeded during the first 15 minutes of incubation which was done stepwise at 55° C. for 5 minutes, 60° C. for 5 minutes, and 65° C. for 5 minutes. The target nucleic acids were then denatured with incubation at 95° C. for 10 minutes after which PCR was run for 45 cycles of 92° C. for 5 seconds, 60° C. for 40 seconds, and 72° C. for 1 second. Reactions were run on a Roche LightCycler® 480 (Roche Applied Science, Indianapolis, Ind., USA) thermocycler. All reactions were performed in triplicate. Note that the 95° C. incubation also activates the HS-P.a. RNase H2 enzyme. After amplification, samples were removed and separated using polyacrylamide gel electrophoresis with an 8% non-denaturing gel and were stained for 10 minutes with 1× GelStar® Nucleic Acid Stain (Lonza, Rockland, Me., USA). Products were visualized by fluorescence with UV excitation.

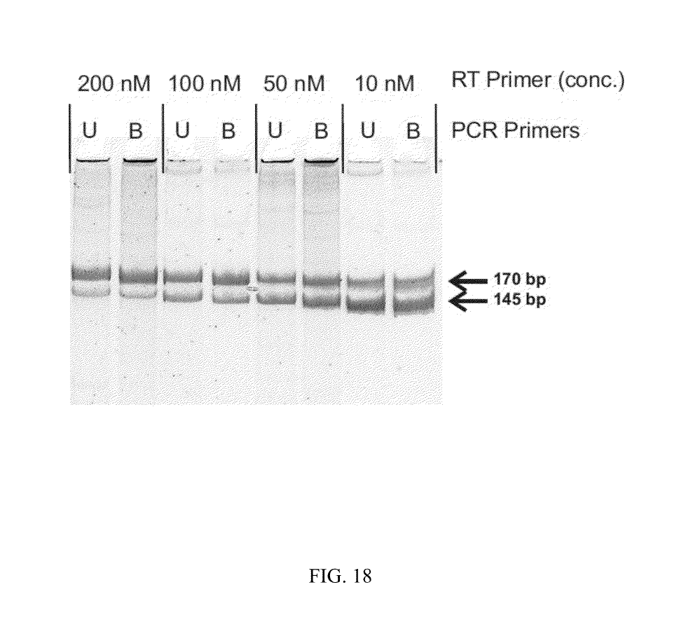

Cycle threshold values of the qPCR 5′-nuclease assay are shown in Table 11 below. As expected, reactions done using blocked primer did not amplify in the absence of RNase H2. All other amplification reactions showed relatively similar Cq values, however the amplified products varied significantly between reactions depending on the RT primer employed, as can be seen in the gel images in FIGS. 18-20.

TABLE 11
Cq values for RT-qPCR of a human SFRS9 amplicon comparing
different designs for external RT primers

| Reaction | | | No | Hot-start |
|---|---|---|---|---|
| RT Primer | | PCR Primers | RNase H | RNase H2 |
| No External RT Primer | | Unmodified | 22.5 | 21.9 |
| | | Blocked | >40 | 27.8 |
| SFRS9-RT | 200 nM | Unmodified | 24.8 | 25.1 |
| | | Blocked | >40 | 22.7 |
| | 100 nM | Unmodified | 24.7 | 23.6 |
| | | Blocked | >40 | 22.0 |
| | 50 nM | Unmodified | 23.4 | 23.7 |
| | | Blocked | >40 | 22.6 |
| | 10 nM | Unmodified | 22.9 | 22.3 |
| | | Blocked | >40 | 23.0 |
| SFRS9-RT-rC | 200 nM | Unmodified | 24.2 | 24.5 |
| | | Blocked | >40 | 22.8 |
| | 100 nM | Unmodified | 23.6 | 23.8 |
| | | Blocked | >40 | 23.1 |
| | 50 nM | Unmodified | 22.9 | 23.2 |
| | | Blocked | >40 | 23.7 |
| | 10 nM | Unmodified | 23.3 | 22.5 |
| | | Blocked | >40 | 22.8 |
| SFRS9-RT-ZEN | 200 nM | Unmodified | 25.5 | 25.3 |
| | | Blocked | >40 | 22.8 |
| | 100 nM | Unmodified | 25.2 | 25.1 |
| | | Blocked | >40 | 22.9 |
| | 50 nM | Unmodified | 24.1 | 23.9 |
| | | Blocked | >40 | 23.1 |
| | 10 nM | Unmodified | 23.5 | 23.5 |
| | | Blocked | >40 | 23.8 |

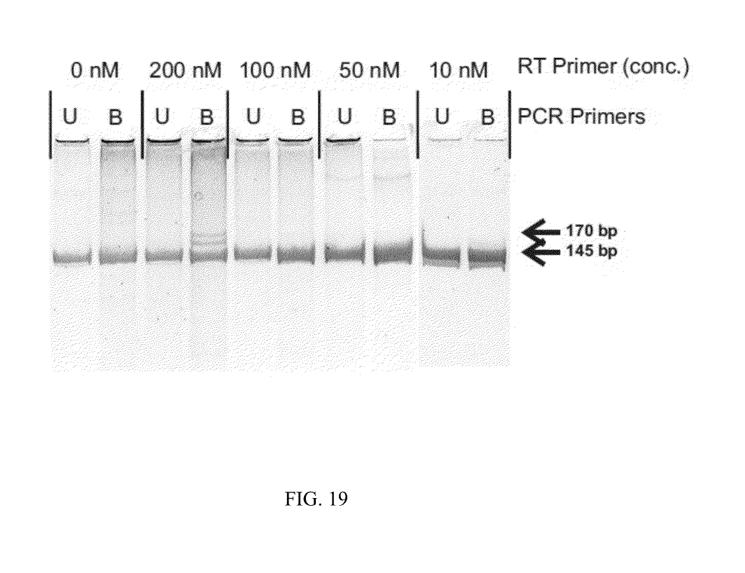

The amplification reactions produced either the desired 145 bp amplicon made from the For and Rev PCR primers (SEQ ID NOs. 17 & 18 or 27 & 28) or an undesired 170 by amplicon made from the For PCR primer (SEQ ID NOs. 17 or 27) and the RT primer (SEQ ID NOs. 29, 30, or 31). Use of the For and Rev PCR primers without an external RT primer produced only the expected 145 bp amplicon (FIG. 19, “0 nM RT Primer” lanes).

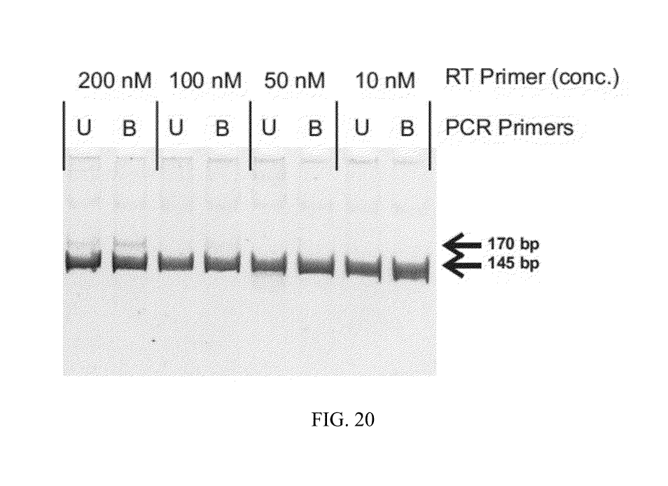

The unmodified RT primer (SEQ ID NO. 31) participated in the PCR reaction, leading to formation of varying amounts of the undesired 170 bp product (FIG. 18). The amount of this product decreased with use of lower concentrations of the RT primer; however, a significant amount of the product remained even when using only 10 nM of the unmodified RT primer. In contrast, the desired 145 bp amplicon was almost exclusively made using the modified RT primer with a central rC RNA residue (SEQ ID NO. 30) (FIG. 19). This oligonucleotide will prime the RT reaction but is degraded by P.a. RNase H2 after heat reactivation and so cannot participate in PCR amplification. Use of lower RT concentrations (50 nM and 10 nM) gave the most robust yields of the desired product with all external primer designs. Use of the modified RT primer containing a central abasic napthyl-azo modifier (SEQ ID NO. 29) (FIG. 20) also produced mostly the desired 145 bp amplicon. This oligonucleotide will prime the RT reaction and remains competent to prime DNA synthesis during PCR, however it can only sustain linear amplification and cannot support exponential amplification since it is defective in template function and the final amplification product does not contain a complete primer-binding site. Use of lower concentrations of this primer also showed the most robust reactions (50 nM and 10 nM).

The 3,4,5,6-tetrahydrophthalic anhydride-modified HS-P.a. RNase H2 permits rhPCR to be performed using blocked For and Rev primers in a single-tube high-temperature RT-qPCR format. Use of an unmodified RT primer results in production of undesired, longer amplification products but use of modified RT primers that can prime RT but cannot participate in PCR results in production of the desired amplicon with high specificity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of copying a target RNA molecule in a sample to produce a cDNA, the method comprising the steps of:
a) treating said sample in a reaction mixture comprising a polymerase, deoxyribonucleoside triphosphates, a buffer, a RNase H enzyme that is reversibly inactive, a first primer, wherein the first primer is blocked at or near the 3′-end of the first primer with a blocking group that is removable with an RNase H enzyme, and wherein the first primer is complementary to the target RNA to hybridize and form a double-stranded product;
b) raising the temperature to activate the RNase H enzyme;
c) hybridizing the first primer with the target RNA at an appropriate temperature;
d) treating the double-stranded product with RNase H to remove the blocking group; and
e) polymerizing the first primer to form a cDNA complementary to the target RNA.

2. The method of claim 1 wherein the reaction mixture further comprises a second primer complementary to the cDNA and wherein the method further comprises hybridizing the second primer to the cDNA and polymerizing the second primer to form an extension product complementary to the cDNA.

3. The method of claim 1 wherein the RNase H enzyme is an RNase H2 enzyme.

4. The method of claim 3 wherein the RNase H2 enzyme is a Pyrococcus abyssi RNase H2 enzyme.

5. The method of claim 1 wherein the polymerase is not a hot-start polymerase.

6. A modified Pyrococcus abyssi RNase H2 protein comprising at least one modified lysine residue of structure I: 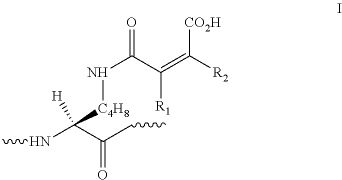
wherein R¹ and R² are independently selected from the group consisting of lower alkyl, lower cycloalkyl, lower alkenyl, lower aryl, lower arylalkyl, lower alkoxy, acyl, or carboalkoxy groups, or together define a lower carbocycle or lower heterocycle, each of R¹ and R² independently optionally substituted with halogen, alkoxy, amino, acyl, carboxy, carboalkoxy, or carbamyl.

7. The modified Pyrococcus abyssi RNase H2 protein of claim 6, wherein one of R¹ and R² is H, and the other of R¹ and R² is CH₃.

8. The modified Pyrococcus abyssi RNase H2 protein of claim 6, wherein one of R¹ and R² is H, and the other of R¹ and R² is CH₂CO₂H

9. The modified Pyrococcus abyssi RNase H2 protein of claim 6, wherein R¹ and R² are CH₃.

10. The modified Pyrococcus abyssi RNase H2 protein of claim 6, wherein R¹ and R² together are butane-1,4-diyl.

11. The modified Pyrococcus abyssi RNase H2 protein of claim 6, wherein the lysine residue is a conserved lysine residue.

12. The modified Pyrococcus abyssi RNase H2 protein of claim 6, wherein about 25 lysine residues are modified.

13. The modified Pyrococcus abyssi RNase H2 protein of claim 6, wherein from about 22 to about 28 lysine residues are modified.

14. The modified Pyrococcus abyssi RNase H2 protein of claim 6, wherein the activity at (temp) is less than about (low temp activity).

15. A kit comprising:
a modified Pyrococcus abyssi RNase H2 protein comprising at least one modified lysine residue of structure I: 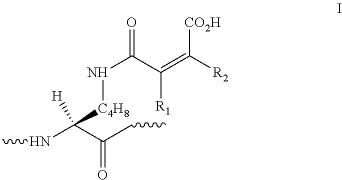
wherein R¹ and R² are independently selected from the group consisting of lower alkyl, lower cycloalkyl, lower alkenyl, lower aryl, lower arylalkyl, lower alkoxy, acyl, or carboalkoxy groups, or together define a lower carbocycle or lower heterocycle, each of R¹ and R² independently optionally substituted with halogen, alkoxy, amino, acyl, carboxy, carboalkoxy, or carbamyl; and
at least one of DNA polymerase or DNA ligase.

16. The kit of claim 15, further comprising an oligonucleotide comprising an RNase H2 cleavage domain.

17. A method for modifying a Pyrococcus abyssi RNase H2 protein, the method comprising:
contacting a Pyrococcus abyssi RNase H2 protein with a compound of formula II: 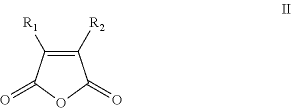
wherein R¹ and R² are independently selected from the group consisting of lower alkyl, lower cycloalkyl, lower alkenyl, lower aryl, lower arylalkyl, lower alkoxy, acyl, or carboalkoxy groups, or together define a lower carbocycle or lower heterocycle, each of R¹ and R² independently optionally substituted with halogen, alkoxy, amino, acyl, carboxy, carboalkoxy, or carbamyl.

18. The method of claim 17, further comprising repeating contacting the Pyrococcus abyssi RNase H2 protein with the compound of formula II.

19. The method of claim 18, wherein the repeating contacting comprises contacting at least a total of three times.

20. The method of claim 18, wherein the repeating contacting comprises contacting at least a total of five times.

21. The method of claim 18, wherein the repeating contacting comprises contacting at least a total of ten times.

22. The method of claim 18, wherein contacting with a compound of formula II comprises contacting with a compound selected from the group consisting of maleic anhydride, citriconyl anhydride, cis-acotinic anhydride, and 3,4,5,6-tetrahydrophthalic anhydride.

23. A method of reactivating a modified Pyrococcus abyssi RNase H2 protein comprising at least one modified lysine residue of structure I: 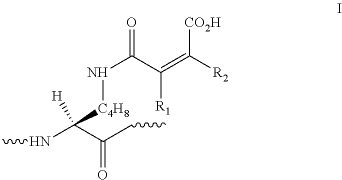
wherein R¹ and R² are independently selected from the group consisting of lower alkyl, lower cycloalkyl, lower alkenyl, lower aryl, lower arylalkyl, lower alkoxy, acyl, or carboalkoxy groups, or together define a lower carbocycle or lower heterocycle, each of R¹ and R² independently optionally substituted with halogen, alkoxy, amino, acyl, carboxy, carboalkoxy, or carbamyl,
the method comprising:
heating the modified Pyrococcus abyssi RNase H2 protein.

24. The method of claim 23, wherein the heating comprises heating to a temperature of at least about 95° C.

25. A method of cleaving a oligonucleotide at a RNase H2 cleavage domain, the method comprising contacting a oligonucleotide comprising an RNase H2 cleavage domain with a modified Pyrococcus abyssi RNase H2 protein comprising at least one modified lysine residue of structure I: 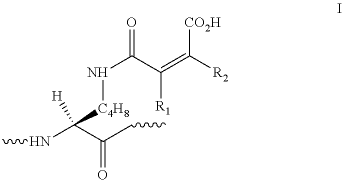
wherein R¹ and R² are independently selected from the group consisting of lower alkyl, lower cycloalkyl, lower alkenyl, lower aryl, lower arylalkyl, lower alkoxy, acyl, or carboalkoxy groups, or together define a lower carbocycle or lower heterocycle, each of R′ and R² independently optionally substituted with halogen, alkoxy, amino, acyl, carboxy, carboalkoxy, or carbamyl,
at a temperature sufficient to reactivate the modified Pyrococcus abyssi RNase H2 protein, thereby cleaving the oligonucleotide.

26. The method of claim 25, wherein the method is a hot-start method.

27. The method of claim 25, wherein the method is a single tube method.

28. The method of claim 25, wherein the method is a step in at least one of a nucleic acid amplification assay, a nucleic acid detection assay, an oligonucleotide ligation assay (OLA), a primer probe assay, a polymerase chain reaction (PCR), a quantitative polymerase chain reaction (qPCR), a reverse-transcriptase polymerase chain reaction (RT-PCR), a ligase chain reaction (LCR), a polynomial amplification method, DNA sequencing method, or an method comprising primer extension.

29. The method of claim 25, wherein contacting a oligonucleotide comprising an RNase H2 cleavage domain comprises contacting a oligonucleotide comprising a single RNA residue or an RNA base replaced with at least one alternative nucleoside.

30. The method of claim 25, wherein contacting an oligonucleotide comprises contacting a duplex oligonucleotide.

31. The method of claim 25, wherein contacting an oligonucleotide comprises contacting a primer for DNA replication.

32. The method of claim 2 for use in discriminating single-nucleotide polymorphisms (SNPs) in a DNA molecule, wherein the first and second primers are discriminatory primers.

33. The method of claim 2 further comprising a third primer, wherein the third primer is modified such that it does not participate in the subsequent amplification reaction.

34. The method of claim 33 wherein the modification creates a cleavable linkage.

35. The method of claim 34 wherein the cleavable linkage is susceptible to chemical cleavage or restriction enzymes.

36. The method of claim 33 wherein the modification comprises a blocking group.

37. The method of claim 36 wherein the blocking group comprises 2′-modified RNA residues, abasic residues, unnatural bases or a non-nucleotide napthyl-azo modifier.
